(19) United States
(12) Patent Application Publication
(10) Pub. No.: US 2009/0019606 A1
(43) Pub. Date: Jan. 15, 2009

(54) Plants Having Increased Yield And A Method For Making The Same

(75) Inventor: Valerie Frankard, Waterloo (BE)

Correspondence Address: CONNOLLY BOVE LODGE & HUTZ, LLP, P O BOX 2207, WILMINGTON, DE, 19899, US

(73) Assignee: CropDesign N.V., Zwijnaarde (BE)

(21) Appl. No.: 12/280,695

(22) PCT Filed: Feb. 27, 2007
(86) PCT No.: PCT/EP07/51842

§ 371 Date: Aug. 26, 2008

(30) Foreign Application Priority Data
Feb. 28, 2006 (EP) 06075457.9

(51) Int. Cl. C12N 15/33; A01H 5/00

(52) U.S. Cl. 800/287; 800/278; 800/298; 536/23.72; 800/320.1; 800/320.2; 800/320.3; 800/320

(57) ABSTRACT

The present invention relates generally to the field of molecular biology and concerns a method for increasing plant yield relative to control plants. More specifically, the present invention concerns a method for increasing plant yield comprising increasing expression in a plant of a nucleic acid sequence encoding a MYB (DNA-binding) domain transcription factor (MYB-TF) polypeptide. In a particular embodiment, the present invention concerns a method for increasing plant yield comprising preferentially increasing expression of a nucleic acid sequence encoding a MYB-TF polypeptide, in the endosperm of a plant seed. The present invention also concerns plants having increased expression of a nucleic acid sequence encoding a MYB-TF polypeptide, as well as plants having preferentially increased expression of a nucleic acid sequence encoding a MYB-TF polypeptide in the endosperm of seeds, which plants have increased yield relative to control plants. The invention also provides constructs useful in the methods of the invention.

The present invention relates generally to the field of molecular biology and concerns a method for increasing plant yield relative to control plants. More specifically, the present invention concerns a method for increasing plant yield comprising increasing expression in a plant of a nucleic acid sequence encoding a MYB (DNA-binding) domain transcription factor (MYB-TF) polypeptide. In a particular embodiment, the present invention concerns a method for increasing plant yield comprising preferentially increasing expression of a nucleic acid sequence encoding a MYB-TF polypeptide, in the endosperm of a plant seed. The present invention also concerns plants having increased expression of a nucleic acid sequence encoding a MYB domain transcription factor polypeptide, as well as plants having preferentially increased expression of a nucleic acid sequence encoding a MYB domain transcription factor polypeptide, in the endosperm of seeds, which plants have increased yield relative to control plants. The invention also provides constructs useful in the methods of the invention.

The ever-increasing world population and the dwindling supply of arable land available for agriculture fuels research towards increasing the efficiency of agriculture. Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic components that may not always result in the desirable trait being passed on from parent plants. Advances in molecular biology have allowed mankind to modify the germplasm of animals and plants. Genetic engineering of plants entails the isolation and manipulation of genetic material (typically in the form of DNA or RNA) and the subsequent introduction of that genetic material into a plant. Such technology has the capacity to deliver crops or plants having various improved economic, agronomic or horticultural traits.

A trait of particular economic interest is yield, and in the case of many plants seed yield. Yield is normally defined as the measurable produce of economic value from a crop. This may be defined in terms of quantity and/or quality. Yield is directly dependent on several factors, for example, the number and size of the organs, plant architecture (for example, the number of branches), seed production and more. Root development, nutrient uptake and stress tolerance may also be important factors in determining yield. Optimizing one of the abovementioned factors may therefore contribute to increasing crop yield. Plant seeds are an important source of human and animal nutrition. Crops such as, corn, rice, wheat, canola and soybean account for over half of the total human caloric intake, whether through direct consumption of the seeds themselves or through consumption of meat products raised on processed seeds. They are also a source of sugars, oils and many kinds of metabolites used in industrial processes. Seeds contain an embryo, the source of new shoots and roots after germination, and an endosperm, the source of nutrients for embryo growth, during germination and early growth of seedlings. The development of a seed involves many genes, and requires the transfer of metabolites from roots, leaves and stems into the growing seed. The endosperm, in particular, assimilates the metabolic precursors of carbohydrates, oils and proteins and synthesizes them into storage macromolecules to fill out the grain. The ability to increase plant seed yield, whether through seed number, seed biomass, seed development, seed filling, or any other seed-related trait would have many applications in agriculture, and even many non-agricultural uses such as in the biotechnological production of substances such as pharmaceuticals, antibodies or vaccines.

MYB domain proteins are transcription factors with a highly conserved DNA-binding domain. The MYB domain was originally described in the oncogene (v-myb) of avian myeloblastosis virus (Klempnauer et al. (1982) Cell: 453-63). MYB proteins contain one to four imperfect direct repeats of a conserved sequence of 50-53 amino acids which encodes a helix-turn-helix structure involved in DNA binding (Rosinski & Atchley (1998) J Mol Evol 46: 74-83). MYB domain transcription factor polypepides have been identified in many higher eucaryotes, including plants (Jiang et al. (2004) Genome Biology 5:R46). MYB domain transcription factor polypeptides constitute one of the largest family of transcription factors in plants (at least 130 in Arabidopsis thaliana), but with little sequence conservation outside of the MYB domain. They have therefore been clustered into subgroups based on conserved motifs identified outside of the MYB coding region (Jiang et al., supra).

One MYB domain transcription factor polypeptide from rice, OsMYB4, was cloned by hybridisation using the corn C1 MYB-encoding nucleic acid molecule as probe (Suzuki et al. (1997) Gene 198: 393-398; NCBI protein accession number BAA23340). The MYB DNA-binding domain of OsMYB4 is formed by two repeats, which are most similar to the second and third repeat of the three repeats (R1, R2 and R3) usually found in animal MYB DNA-binding domains, and is therefore part of the R2R3-type MYB family of polypeptides. OsMYB4 mRNA levels were preferentially observed in developing seeds, it was therefore postulated that OsMYB4 plays a role in seed maturation (Suzuki et al., supra).

WO 03/007699 describes a nucleic acid sequence encoding an OsMYB4 transcription factor. Also described are methods for using the polynucleotide to alter resistance or tolerance of plants to stress, to alter biological pathways and to alter gene expression.

US patent applications 2004/0045049 and 2004/0019927 provide nucleic acid sequences encoding an OsMYB4 transcription factor and its Arabidopsis ortholog (referred to in the applications as G211). Overexpression of G211 in Arabidopsis was reported to have an effect on leaf and inflorescence development, giving small bushy plants with reduced seed yield compared to wild type controls.

Surprisingly, it has now been found that increasing expression in a plant of a nucleic acid sequence encoding a MYB (DNA-binding) domain transcription factor (MYB-TF) polypeptide gives plants having increased yield relative to control plants. It has also been found that preferentially increasing expression of a nucleic acid sequence encoding a MYB-TF polypeptide in the endosperm of a plant seed, gives plants having increased yield relative to control plants.

According to the present invention, there is provided a method for increasing plant yield relative to control plants, comprising increasing expression in a plant of a nucleic acid sequence encoding MYB-TF polypeptide. In a particular embodiment, there is provided a method for increasing plant yield relative to control plants, comprising preferentially increasing expression of a nucleic acid sequence encoding MYB-TF polypeptide, in the endosperm of a plant seed.

The terms “polypeptide” and “protein” are used interchangeably herein and refer to amino acids in a polymeric form of any length.

The terms “polynucleotide(s)”, “nucleic acid sequence(s)”, “nucleotide sequence(s)” are used interchangeably herein and refer to nucleotides, either ribonucleotides or deoxyribonucleotides or a combination of both, in a polymeric form of any length.

The choice of a control plant is a routine part of an experimental setup and may include corresponding wild type plants or corresponding plants without the gene of interest. A <<control plant>> is typically of the same plant species or even of the same variety as the plant to be assessed. The control plant may also be a nullizygote of the plant to be assessed. A “control plant” as used herein refers not only to whole plants, but also to plant parts, including seeds and seed parts.

The term “increased yield” as defined herein is taken to mean an increase in biomass (weight) of one or more parts of a plant, which may include aboveground (harvestable) parts and/or (harvestable) parts below ground.

In particular, such harvestable parts are seeds, and performance of the methods of the invention results in plants having increased seed yield relative to the seed yield of control plants.

Increased seed yield may manifest itself as one or more of the following: a) an increase in seed biomass (total seed weight) which may be on an individual seed basis and/or per plant and/or per hectare or acre; b) increased number of flowers per plant; c) increased number of (filled) seeds; d) increased seed filling rate, which is expressed as the ratio between the number of filled seeds divided by the total number of seeds; e) increased harvest index, which is expressed as a ratio of the yield of harvestable parts, such as seeds, divided by the total biomass; and f) increased thousand kernel weight (TKW), which is extrapolated from the number of filled seeds counted and their total weight. An increased TKW may result from an increased seed size and/or seed weight, and may also result from an increase in embryo and/or endosperm size.

An increase in seed yield may also be manifested as an increase in seed size and/or seed volume. Furthermore, an increase in seed yield may also manifest itself as an increase in seed area and/or seed length and/or seed width and/or seed perimeter. Increased yield may also result in modified architecture, or may occur because of modified architecture

Taking corn as an example, a yield increase may be manifested as one or more of the following: increase in the number of plants per hectare or acre, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others. Taking rice as an example, a yield increase may manifest itself as an increase in one or more of the following: number of plants per hectare or acre, number of panicles per plant, number of spikelets per panicle, number of flowers (florets) per panicle (which is expressed as a ratio of the number of filled seeds over the number of primary panicles), increase in the seed filling rate (which is the number of filled seeds divided by the total number of seeds and multiplied by 100), increase in thousand kernel weight, among others.

According to a preferred feature of the present invention, performance of the methods of the invention gives plants having increased seed yield relative to control plants. Therefore according to the present invention, there is provided a method for increasing seed yield in plants relative to the seed yield of control plants, the method comprising increasing expression in a plant of a nucleic acid sequence encoding MYB domain transcription factor polypeptide. In a particular embodiment, there is provided a method for increasing seed yield in plants relative to the seed yield of control plants, the method comprising preferentially increasing expression of a nucleic acid sequence encoding MYB domain transcription factor polypeptide, in the endosperm of a plant seed.

Since the transgenic plants according to the present invention have increased yield, it is likely that these plants exhibit an increased growth rate (during at least part of their life cycle), relative to the growth rate of corresponding wild type plants at a corresponding stage in their life cycle. The increased growth rate may be specific to one or more parts of a plant (including seeds), or may be throughout substantially the whole plant. A plant having an increased growth rate may even exhibit early flowering. The increase in growth rate may take place at one or more stages in the life cycle of a plant or during substantially the whole plant life cycle. Increased growth rate during the early stages in the life cycle of a plant may reflect enhanced vigour (increased seedling vigor at emergence). The increase in growth rate may alter the harvest cycle of a plant allowing plants to be sown later and/or harvested sooner than would otherwise be possible. If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of rice plants followed by, for example, the sowing and optional harvesting of soy bean, potato or any other suitable plant). Harvesting additional times from the same rootstock in the case of some crop plants may also be possible. Altering the harvest cycle of a plant may lead to an increase in annual biomass production per square meter (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

Performance of the methods of the invention gives plants having an increased growth rate. Therefore, according to the present invention, there is provided a method for increasing the growth rate of plants, which method comprises increasing expression in a plant of a nucleic acid sequence encoding a MYB-TF polypeptide. In a particular embodiment, there is provided a method for increasing the growth rate of plants, which method comprises preferentially increasing expression of a nucleic acid sequence encoding a MYB-TF polypeptide, in the endosperm of a plant seed.

An increase in yield and/or growth rate occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35% or 30%, preferably less than 25%, 20% or 15%, more preferably less than 14%, 13%, 12%, 11% or 10% or less in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the everyday biotic and/or abiotic (environmental) stresses to which a plant is exposed. Abiotic stresses may be due to drought or excess water, anaerobic stress, salt stress, nutrient depletion, chemical toxicity, oxidative stress and hot, cold or freezing temperatures. The abiotic stress may be an osmotic stress caused by a water stress (particularly due to drought), salt stress, oxidative stress or an ionic stress. Biotic stresses are typically those stresses caused by pathogens, such as bacteria, viruses, fungi and insects.

In particular, the methods of the present invention may be performed under non-stress conditions or under conditions of mild drought to give plants having increased seed yield relative to control plants. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signaling pathways and cellular responses, such as the production of stress proteins, up-regulation of anti-oxidants, accumulation of compatible solutes and growth arrest. The term “non-stress” conditions as used herein are those environmental conditions that allow optimal growth of plants. Persons skilled in the art are aware of normal soil conditions and climatic conditions for a given location.

Performance of the methods of the invention gives plants grown under non-stress conditions or under mild drought conditions increased seed yield relative to control plants grown under comparable conditions. Therefore, according to the present invention, there is provided a method for increasing plant yield of plants grown under non-stress conditions or under mild drought conditions, which method comprises increasing expression in a plant of a nucleic acid sequence encoding a MYB-TF polypeptide as defined above. In a particular embodiment, the present invention concerns a method for increasing plant yield of plants grown under non-stress conditions or under mild drought conditions, comprising preferentially increasing expression of a nucleic acid sequence encoding a MYB-TF polypeptide, in the endosperm of a plant seed.

The methods of the invention are advantageously applicable to any plant.

The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid sequence of interest. The term “plant” also encompasses plant cells, suspension cultures, callus tissue, embryos, meristematic regions, gametophytes, sporophytes, pollen and microspores, again wherein each of the aforementioned comprises the gene/nucleic acid sequence of interest.

Plants that are particularly useful in the methods of the invention include all plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Triticosecale rimpaui, Triticum spp. (e.g. Triticum aestivum, Triticum durum, Triticum turgidum, Triticum hybernum, Triticum macha, Triticum sativum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

According to a preferred embodiment of the present invention, the plant is a crop plant. Examples of crop plants include soybean, sunflower, canola, alfalfa, rapeseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, triticale, sorghum and oats.

The term “MYB-TF polypeptide” as defined herein refers to any polypeptide comprising from N-terminus to C-terminus (i) an R2R3 MYB domain comprising two MYB repeats; and (ii) a MYB4 domain having in increasing order of preference at least 55%, 60%, 65%, 70%, 75%, 80%, 85%, 90%, 95%, 98%, 99% or more sequence identity to the MYB4 domain represented by any one or more of: SEQ ID NO: 38, SEQ ID NO: 27, or SEQ ID NO: 28.

Examples of MYB-TF polypeptides as defined hereinabove are given in Table A of Example 1.

Alternatively or additionally, a “MYB-TF polypeptide” as defined herein refers to any polypeptide sequence which when used in the construction of a MYB phylogenetic tree, such as the one depicted in FIG. 3, tends to cluster with the group of polypeptide sequences comprising the polypeptide sequences as represented by SEQ ID NO: 2 and SEQ ID NO: 4 (FIG. 3, bold arrow; the circle shows the origin of the group on the tree) rather than with any other group. A preferred method for generating a phylogenetic tree is described by Kranz et al. (1998) Plant Journal 16(2): 263-276).

A person skilled in the art could readily determine whether any polypeptide sequence in question falls within the definition of a “MYB-TF polypeptide” using known techniques and software for the making of such a phylogenetic tree, such as a GCG, EBI or CLUSTAL package, using default parameters. Any sequence clustering within the group comprising the polypeptide sequences as represented by SEQ ID NO: 2 and SEQ ID NO: 4 would be considered to fall within the aforementioned definition of a “MYB-TF polypeptide”, and would be considered suitable for use in the methods of the invention.

Alternatively or additionally, a “MYB-TF polypeptide” as defined herein refers to any polypeptide sequence with in increasing order of preference at least 35%, 40%, 45%, 50%, 55%, 60%, 65%, 70%, 75%, 80%, 85%, 90%, 95%, 98%, 99% or more sequence identity to the polypeptide sequence as represented by SEQ ID NO: 2.

Homologues of a MYB-TF polypeptide may also be used to perform the methods of invention. “Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived.

A deletion refers to removal of one or more amino acids from a protein.

An insertion refers to one or more amino acid residues being introduced into a predetermined site in a protein. Insertions may comprise N-terminal and/or C-terminal fusions as well as intra-sequence insertions of single or multiple amino acids. Generally, insertions within the amino acid sequence will be smaller than N- or C-terminal fusions, of the order of about 1 to 10 residues. Examples of N- or C-terminal fusion proteins or peptides include the binding domain or activation domain of a transcriptional activator as used in the yeast two-hybrid system, phage coat proteins, (histidine)-6-tag, glutathione S-transferase-tag, protein A, maltose-binding protein, dihydrofolate reductase, Tag•100 epitope, c-myc epitope, FLAG®-epitope, lacZ, CMP (calmodulin-binding peptide), HA epitope, protein C epitope and VSV epitope.

A substitution refers to replacement of amino acids of the protein with other amino acids having similar properties (such as similar hydrophobicity, hydrophilicity, antigenicity, propensity to form or break α-helical structures or β-sheet structures). Amino acid substitutions are typically of single residues, but may be clustered depending upon functional constraints placed upon the polypeptide; insertions will usually be of the order of about 1 to 10 amino acid residues. The amino acid substitutions are preferably conservative amino acid substitutions. Conservative substitution tables are well known in the art (see for example Creighton (1984) Proteins. W.H. Freeman and Company and Table 1 below).

TABLE 1
Examples of conserved amino acid substitutions

| Residue | Conservative Substitutions |
|---|---|
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | Leu, Val |
| Leu | Ile; Val |
| Lys | Arg; Gln |
| Met | Leu; Ile |
| Phe | Met; Leu; Tyr |
| Ser | Thr; Gly |
| Thr | Ser; Val |
| Trp | Tyr |
| Tyr | Trp; Phe |
| Val | Ile; Leu |

Amino acid substitutions, deletions and/or insertions may readily be made using peptide synthetic techniques well known in the art, such as solid phase peptide synthesis and the like, or by recombinant DNA manipulation. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols.

Homologues (or homologous proteins) may readily be identified using routine techniques well known in the art, such as by sequence alignment. Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the alignment of two complete sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information. Homologues may readily be identified using, for example, the ClustalW multiple sequence alignment algorithm (version 1.83) available at GenomeNet service at the Kyoto University Bioinformatics Center, with the default pairwise alignment parameters, and a scoring method in percentage. Minor manual editing may be performed to optimise alignment between conserved motifs, as would be apparent to a person skilled in the art.

Homologues also include orthologues and paralogues, which encompass evolutionary concepts used to describe ancestral relationships of genes. Paralogues are genes within the same species that have originated through duplication of an ancestral gene and orthologues are genes from different organisms that have originated through speciation.

Orthologues and paralogues may easily be found by performing a so-called reciprocal blast search. This may be done by a first BLAST involving BLASTing a query sequence (for example, SEQ ID NO: 1 or SEQ ID NO: 2) against any sequence database, such as the publicly available NCBI database. BLASTN or TBLASTX (using standard default values) may be used when starting from a nucleotide sequence and BLASTP or TBLASTN (using standard default values) may be used when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 1 or SEQ ID NO: 2 the second BLAST would therefore be against Oryza sequences). The results of the first and second BLASTs are then compared. A paralogue is identified if a high-ranking hit from the second BLAST is from the same species as from which the query sequence is derived; an orthologue is identified if a high-ranking hit is not from the same species as from which the query sequence is derived. High-ranking hits are those having a low E-value. The lower the E-value, the more significant the score (or in other words the lower the chance that the hit was found by chance). Computation of the E-value is well known in the art. In the case of large families, ClustalW may be used, followed by a neighbour joining tree, to help visualize clustering of related genes and to identify orthologues and paralogues. In addition to E-values, comparisons are also scored by percentage identity. Percentage identity refers to the number of identical nucleotides (or amino acids) between the two compared nucleic acid (or polypeptide) sequences over a particular length. Preferably, MYB-TF polypeptide homologues have in increasing order of preference at least 25%, 30%, 40%, 50%, 60%, 70%, 80%, 85%, 90%, 95%, 96%, 97%, 98%, 99% or more sequence identity or similarity (functional identity) to an unmodified MYB-TF polypeptide as represented by SEQ ID NO: 2. Percentage identity between MYB-TF polypeptide homologues outside of the MYB domain is reputedly low. Preferably, MYB-TF polypeptide homologues are as represented by any of the polypeptide sequences given in Table A, or to any orthologue or paralogue of any of the SEQ ID NOs given in Table A.

The MYB-TF polypeptide may be a derivative of SEQ ID NO: 2. “Derivatives” include peptides, oligopeptides, polypeptides which may, compared to the amino acid sequence of the naturally-occurring form of the protein, such as the one presented in SEQ ID NO: 2, comprise substitutions of amino acids with non-naturally occurring amino acid residues, or additions of non-naturally occurring amino acid residues. Derivatives of any of the polypeptide sequences given in Table A, or to any orthologue or paralogue of any of the SEQ ID NOs given in Table A are further examples which may be suitable for use in the methods of the invention

“Derivatives” of a protein also encompass peptides, oligopeptides, polypeptides which may comprise naturally occurring altered (glycosylated, acylated, ubiquinated, prenylated, phosphorylated, myristoylated, sulphated etc) or non-naturally altered amino acid residues compared to the amino acid sequence of a naturally-occurring form of the polypeptide. A derivative may also comprise one or more non-amino acid substituents or additions compared to the amino acid sequence from which it is derived, for example a reporter molecule or other ligand, covalently or non-covalently bound to the amino acid sequence, such as a reporter molecule which is bound to facilitate its detection, and non-naturally occurring amino acid residues relative to the amino acid sequence of a naturally-occurring protein.

The term “domain” means a set of amino acids conserved at specific positions along an alignment of sequences (performed as described hereinabove) of evolutionarily related proteins. While amino acids at other positions can vary between homologues, amino acids that are highly conserved at specific positions indicate amino acids that are essential in the structure, the stability, or the activity of a protein. Because they are identified by their high degree of conservation in aligned sequences of a family of protein homologues, they can be used as identifiers to determine if a polypeptide with a newly determined sequence belongs to a previously identified polypeptide family.

The MYB domain (comprising MYB repeats) in a MYB-TF polypeptide may be identified using specialised databases e.g. SMART (Schultz et al. (1998) Proc. Natl. Acad. Sci. USA 95, 5857-5864; Letunic et al. (2002) Nucleic Acids Res 30, 242-244; hosted by the EMBL at Heidelberg, Germany), InterPro (Mulder et al., (2003) Nucl. Acids. Res. 31, 315-318; hosted by the European Bioinformatics Institute (EBI) in the United Kingdom), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAIPress, Menlo Park; Hulo et al., Nucl. Acids. Res. 32:D134-D137, (2004), the ExPASy proteomics server is provided as a service to the scientific community (hosted by the Swiss Institute of Bioinformatics (SIB) in Switzerland) or Pfam (Bateman et al., Nucleic Acids Research 30(1): 276-280 (2002), hosted by the Sanger Institute in the United Kingdom). The MYB domain comprises up to four imperfect repeats, each forming a helix-turn-helix structure of about 53 amino acids. MYB repeats are characterized by regularly spaced tryptophan residues.

The MYB4 domain as represented by SEQ ID NO: 38 ((D/N)(D/A)XF(S/T/P/-)SFL(N/D)(S/A)L(I/M)N(E/D), as represented by SEQ ID NO: 27 ((D/N)(D/A)XF(S/T/-)SFL(N/D)SL(I/M)N(E/D), where X is any amino and where - is no amino acid), and/or as represented by SEQ ID NO: 28 (DDDFSSFLDSLIND), may be identified using methods for the alignment of sequences for comparison as described hereinabove. In some instances, the default parameters may be adjusted to modify the stringency of the search. For example using BLAST, the statistical significance threshold (called “expect” value) for reporting matches against database sequences may be increased to show less stringent matches. This way, short nearly exact matches may be identified, including the MYB4 domain of SEQ ID NO: 38 and/or the MYB4 domain of SEQ ID NO: 27 with no changes, or with one or more conservative change at any position, or with one, two or three non-conservative change(s) at any position; or with one deletion at any position. For example, the MYB4 domain of the Oryza sativa MYB-TF polypeptide as represented by SEQ ID NO: 2, is given in SEQ ID NO: 28 (DDDFSSFLDSLIND). Preferably, MYB-TF polypeptides useful in performing the methods of the invention comprise a MYB4 domain having in increasing order of preference at least 55%, 60%, 65%, 70%, 75%, 80%, 85%, 90%, 95%, 98%, 99% or more sequence identity to the MYB4 domain represented by any one or more of: SEQ ID NO: 38, SEQ ID NO: 27, or SEQ ID NO: 28.

Furthermore, MYB-TF polypeptides (at least in their native form) typically have DNA-binding activity and an activation domain. A person skilled in the art may easily determine the presence of an activation domain and DNA-binding activity using routine techniques and procedures. Proteins interacting with MYB-TF polypeptides (as, for example, in transcriptional complexes) may easily be identified using standard techniques for a person skilled in the art.

Examples of nucleic acid sequences encoding MYB-TF polypeptides include but are not limited to those represented by any of the nucleic acid sequences given in Table A, or to any nucleic acid sequence encoding orthologue or paralogue of any of the SEQ ID NOs given in Table A. Variants of nucleic acid sequences encoding MYB-TF polypeptides may be suitable for use in the methods of the invention. Suitable variants include portions of nucleic acid sequences encoding MYB-TF polypeptides and/or nucleic acid sequences capable of hybridising with genes/nucleic acid sequences encoding MYB-TF polypeptides. Further variants include splice variants and allelic variants of nucleic acid sequences encoding MYB-TF polypeptides.

The term “portion” as defined herein refers to a piece of DNA encoding a polypeptide comprising from N-terminus to C-terminus (i) an R2R3 MYB domain comprising two MYB repeats; and (ii) a MYB4 domain having in increasing order of preference at least 55%, 60%, 65%, 70%, 75%, 80%, 85%, 90%, 95%, 98%, 99% or more sequence identity to the MYB4 domain represented by any one or more of: SEQ ID NO: 38, SEQ ID NO: 27, or SEQ ID NO: 28. A portion may be prepared, for example, by making one or more deletions to a nucleic acid sequence encoding a MYB-TF polypeptide. The portions may be used in isolated form or they may be fused to other coding (or non coding) sequences in order to, for example, produce a protein that combines several activities. When fused to other coding sequences, the resulting polypeptide produced upon translation may be bigger than that predicted for the MYB-TF portion. Preferably, the portion codes for a polypeptide with substantially the same biological activity as the MYB-TF polypeptide of SEQ ID NO: 2.

Preferably, the portion is a portion of a nucleic acid sequence as represented by any one of the nucleic acid sequences given in Table A, or to any nucleic acid sequence encoding an orthologue or paralogue of any of the SEQ ID NOs given in Table A. Most preferably the portion is a portion of a nucleic acid sequence as represented by SEQ ID NO: 1.

Another variant of a nucleic acid sequence encoding a MYB-TF polypeptide, useful in the methods of the present invention, is a nucleic acid sequence capable of hybridising under reduced stringency conditions, preferably under stringent conditions, with a probe derived from the nucleic acid sequence as defined hereinbefore, which hybridising sequence encodes a polypeptide comprising from N-terminus to C-terminus (i) an R2R3 MYB domain comprising two MYB repeats; and (ii) a MYB4 domain having in increasing order of preference at least 55%, 60%, 65%, 70%, 75%, 80%, 85%, 90%, 95%, 98%, 99% or more sequence identity to the MYB4 domain represented by any one or more of: SEQ ID NO: 38, SEQ ID NO: 27, or SEQ ID NO: 28.

Preferably, the hybridising sequence is one that is capable of hybridising to a nucleic acid sequence as represented by (or to probes derived from) any of the nucleic acid sequences given in Table A, or to any nucleic acid sequence encoding an orthologue or paralogue of any of the SEQ ID NOs given in Table A, or to a portion of any of the aforementioned sequences (the target sequence). Most preferably the hybridising sequence is capable of hybridising to SEQ ID NO: 1 (or to probes derived therefrom). Probes are generally less than 1000 bp in length, preferably less than 500 bp in length. Commonly, probe lengths for DNA-DNA hybridisations such as Southern blotting, vary between 100 and 500 bp, whereas the hybridising region in probes for DNA-DNA hybridisations such as in PCR amplification generally are shorter than 50 but longer than 10 nucleotides.

The term “hybridisation” as defined herein is a process wherein substantially homologous complementary nucleotide sequences anneal to each other. The hybridisation process can occur entirely in solution, i.e. both complementary nucleic acid molecules are in solution. The hybridisation process can also occur with one of the complementary nucleic acid molecules immobilised to a matrix such as magnetic beads, Sepharose beads or any other resin. The hybridisation process can furthermore occur with one of the complementary nucleic acid molecules immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acid molecules.

The term “stringency” refers to the conditions under which a hybridisation takes place. The stringency of hybridisation is influenced by conditions such as temperature, salt concentration, ionic strength and hybridisation buffer composition. Generally, low stringency conditions are selected to be about 30° C. lower than the thermal melting point (Tₘ) for the specific sequence at a defined ionic strength and pH. Medium stringency conditions are when the temperature is 20° C. below Tₘ, and high stringency conditions are when the temperature is 10° C. below Tₘ. High stringency hybridisation conditions are typically used for isolating hybridising sequences that have high sequence similarity to the target nucleic acid sequence. However, nucleic acid sequences may deviate from each other and still encode a substantially identical polypeptide, due to the degeneracy of the genetic code. Therefore medium stringency hybridisation conditions may sometimes be needed to identify such nucleic acid molecules.

The Tₘis the temperature under defined ionic strength and pH, at which 50% of the target sequence hybridises to a perfectly matched probe. The Tₘis dependent upon the solution conditions and the base composition and length of the probe. For example, longer sequences hybridise specifically at higher temperatures. The maximum rate of hybridisation is obtained from about 16° C. up to 32° C. below Tₘ. The presence of monovalent cations in the hybridisation solution reduce the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tₘ decreases about 1° C. per % base mismatch. The Tₘ may be calculated using the following equations, depending on the types of hybrids:
DNA-DNA hybrids (Meinkoth and Wahl, Anal. Biochem., 138: 267-284, 1984):

Tₘ=81.5° C.+16.6x log [Na⁺]^{a}+0.41x%[G/C^{b}]−500x[L^{c}]⁻¹−0.61x% formamide
DNA-RNA or RNA-RNA hybrids:

Tₘ=79.8+18.5(log₁₀[Na⁺]^{a})+0.58 (%G/C^{b})+11.8 (%G/C^{b})²−820/L^{c}
oligo-DNA or oligo-RNA^{d} hybrids:
   For <20 nucleotides: Tₘ=2 (lₙ)
   For 20-35 nucleotides: Tₘ=22+1.46 (lₙ)
   ^{a}or for other monovalent cation, but only accurate in the 0.01-0.4 M range.
   ^{b}only accurate for % GC in the 30% to 75% range.
   ^{c}L=length of duplex in base pairs.
   ^{d}Oligo, oligonucleotide; lₙ, effective length of primer=2×(no. of G/C)+(no. of A/T).

Non-specific binding may be controlled using any one of a number of known techniques such as, for example, blocking the membrane with protein containing solutions, additions of heterologous RNA, DNA, and SDS to the hybridisation buffer, and treatment with Rnase. For non-homologous probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridisation and which will either maintain or change the stringency conditions.

Besides the hybridisation conditions, specificity of hybridisation typically also depends on the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. A positive hybridisation gives a signal that is at least twice of that of the background. Generally, suitable stringent conditions for nucleic acid hybridisation assays or gene amplification detection procedures are as set forth above. More or less stringent conditions may also be selected. The skilled artisan is aware of various parameters which may be altered during washing and which will either maintain or change the stringency conditions.

For example, typical high stringency hybridisation conditions for DNA hybrids longer than 50 nucleotides encompass hybridisation at 65° C. in 1×SSC or at 42° C. in 1×SSC and 50% formamide, followed by washing at 65° C. in 0.3×SSC. Examples of medium stringency hybridisation conditions for DNA hybrids longer than 50 nucleotides encompass hybridisation at 50° C. in 4×SSC or at 40° C. in 6×SSC and 50% formamide, followed by washing at 50° C. in 2×SSC. The length of the hybrid is the anticipated length for the hybridising nucleic acid molecule. When known nucleic acid sequences are hybridised, the hybrid length may be determined by aligning the sequences and identifying the conserved regions described herein. 1×SSC is 0.15M NaCl and 15 mM sodium citrate; the hybridisations and washes may additionally include 5×Denhardt's reagent, 0.5-1.0% SDS, 100 μg/ml denatured, fragmented salmon sperm DNA, 0.5% sodium pyrophosphate.

For the purposes of defining the level of stringency, reference can conveniently be made to Sambrook et al. (2001) Molecular Cloning: a laboratory manual, 3^{rd}Edition Cold Spring Harbor Laboratory Press, CSH, New York, or to Current Protocols in Molecular Biology, John Wiley & Sons, N.Y. (1989 and yearly updates).

The MYB-TF polypeptide may be encoded by an alternative splice variant. The term “alternative splice variant” as used herein encompasses variants of a nucleic acid sequence in which selected introns and/or exons have been excised, replaced, displaced or added, or in which introns have been shortened or lengthened. Such variants will be ones in which the substantial biological activity of the protein is retained, which may be achieved by selectively retaining functional segments of the protein. Such splice variants may be found in nature or may be manmade. Methods for making such splice variants are well known in the art.

Preferred splice variants are splice variants of the nucleic acid sequence encoding a MYB-TF polypeptide comprising from N-terminus to C-terminus (i) an R2R3 MYB domain comprising two MYB repeats; and (ii) a MYB4 domain having in increasing order of preference at least 55%, 60%, 65%, 70%, 75%, 80%, 85%, 90%, 95%, 98%, 99% or more sequence identity to the MYB4 domain represented by any one or more of: SEQ ID NO: 38, SEQ ID NO: 27, or SEQ ID NO: 28. Further preferred splice variants of nucleic acid sequences encoding MYB-TF polypeptides comprising features as defined hereinabove are splice variants of a nucleic acid sequence as given in Table A, or to any nucleic acid sequence encoding an orthologue or paralogue of any of the SEQ ID NOs given in Table A. Most preferred is a splice variant of a nucleic acid sequence as represented by SEQ ID NO: 1.

The MYB-TF polypeptide may also be encoded by an allelic variant of a nucleic acid sequence encoding a polypeptide comprising from comprising from N-terminus to C-terminus (i) an R2R3 MYB domain comprising two MYB repeats; and (ii) a MYB4 domain having in increasing order of preference at least 55%, 60%, 65%, 70%, 75%, 80%, 85%, 90%, 95%, 98%, 99% or more sequence identity to the MYB4 domain represented by any one or more of: SEQ ID NO: 38, SEQ ID NO: 27, or SEQ ID NO: 28.

Preferred allelic variants of nucleic acid sequences encoding MYB-TF polypeptides comprising features as defined hereinabove are allelic variants of a nucleic acid sequence as given in Table A, or to any nucleic acid sequence encoding an orthologue or paralogue of any of the SEQ ID NOs given in Table A. Most preferred is an allelic variant of a nucleic acid sequence as represented by SEQ ID NO: 1. Allelic variants exist in nature, and encompassed within the methods of the present invention is the use of these natural alleles. Allelic variants encompass Single Nucleotide Polymorphisms (SNPs), as well as Small Insertion/Deletion Polymorphisms (INDELs). The size of INDELs is usually less than 100 bp. SNPs and INDELs form the largest set of sequence variants in naturally occurring polymorphic strains of most organisms.

Directed evolution (or gene shuffling) may also be used to generate variants of nucleic acid sequences encoding MYB-TF polypeptides. This consists of iterations of DNA shuffling followed by appropriate screening and/or selection to generate variants of nucleic acid sequences or portions thereof encoding MYB-TF polypeptides or homologues or portions thereof having an modified biological activity (Castle et al., (2004) Science 304(5674): 1151-4; U.S. Pat. Nos. 5,811,238 and 6,395,547).

Site-directed mutagenesis may be used to generate variants of nucleic acid sequences encoding MYB-TF polypeptides. Several methods are available to achieve site-directed mutagenesis, the most common being PCR based methods (Current Protocols in Molecular Biology. Wiley Eds.).

Nucleic acid sequences encoding MYB-TF polypeptides may be derived from any natural or artificial source. The nucleic acid sequence may be modified from its native form in composition and/or genomic environment through deliberate human manipulation. In one embodiment, the MYB-TF nucleic acid sequence is of plant origin, preferably from a monocotyledonous plant, further preferably from the family Poaceae, more preferably from the genus Oryza, most preferably the nucleic acid sequence is from Oryza sativa.

The increased expression of a nucleic acid sequence encoding a MYB-TF polypeptide may be achieved by introducing a genetic modification, preferably in the locus of a MYB-TF gene, in a plant. The locus of a gene as defined herein is taken to mean a genomic region, which includes the gene of interest and 10 KB up- or downstream of the coding region. In a particular embodiment, the expression of a nucleic acid sequence encoding a MYB-TF polypeptide is preferentially increased in the endosperm of a plant seed.

The genetic modification may be introduced, for example, by any one or more of the following methods: T-DNA activation, TILLING and homologous recombination or by introducing and expressing in a plant, a nucleic acid sequence encoding a MYB-TF polypeptide. The expression of the nucleic acid encoding a MYB-TF polypeptide is increased. In a particular embodiment, the expression a nucleic acid sequence encoding a MYB-TF polypeptide is preferentially increased in the endosperm of a plant seed. Following introduction of the genetic modification, there follows an optional step of selecting for increased expression in the plant of a nucleic acid sequence encoding a MYB-TF polypeptide, which increased expression gives plants having increased yield relative to control plants. In a particular embodiment, following introduction of the genetic modification, there follows an optional step of selecting for preferentially increased expression of a nucleic acid sequence encoding a MYB-TF polypeptide in the endosperm of a plant seed, which increased expression gives plants having increased yield relative to control plants.

T-DNA activation tagging (Hayashi et al. Science (1992) 1350-1353) involves insertion of T-DNA, usually containing a promoter (may also be a translation enhancer or an intron), in the genomic region of the gene of interest or 10 kb up- or downstream of the coding region of a gene in a configuration such that the promoter directs expression of the targeted gene. Typically, regulation of expression of the targeted gene by its natural promoter is disrupted and the gene falls under the control of the newly introduced promoter. The promoter is typically embedded in a T-DNA. This T-DNA is randomly inserted into the plant genome, for example, through Agrobacterium infection and leads to modified expression of genes near the inserted T-DNA. The resulting transgenic plants show dominant phenotypes due to modified expression of genes close to the introduced promoter. The promoter to be introduced may be any promoter capable of increasing expression in a plant of the nucleic acid sequence encoding a MYB-TF polypeptide. In a particular embodiment, the promoter of choice preferentially increases expression in the endosperm of a plant seed.

A genetic modification may also be introduced in the locus of a gene encoding a MYB-TF polypeptide using the technique of TILLING (Targeted Induced Local Lesions In Genomes). This is a mutagenesis technology useful to generate and/or identify a nucleic acid sequence encoding a MYB-TF polypeptide with modified expression and/or activity. TILLING also allows selection of plants carrying such mutant variants. These mutant variants may exhibit modified expression, either in strength or in location or in timing (if the mutations affect the promoter for example). These mutant variants may exhibit higher MYB-TF activity than that exhibited by the gene in its natural form. These mutant variants may exhibit TILLNG combines high-density mutagenesis with high-throughput screening methods. The steps typically followed in TILLING are: (a) EMS mutagenesis (Redei GP and Koncz C (1992) In Methods in Arabidopsis Research, Koncz C, Chua N H, Schell J, eds. Singapore, World Scientific Publishing Co, pp. 16-82; Feldmann et al., (1994) In Meyerowitz E M, Somerville C R, eds, Arabidopsis. Cold Spring Harbor Laboratory Press, Cold Spring Harbor, N.Y., pp 137-172; Lightner J and Caspar T (1998) In J Martinez-Zapater, J Salinas, eds, Methods on Molecular Biology, Vol. 82. Humana Press, Totowa, N.J., pp 91-104); (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product. Methods for TILLING are well known in the art (McCallum et al., (2000) Nat Biotechnol 18: 455-457; reviewed by Stemple (2004) Nat Rev Genet 5(2): 145-50). Plants carrying such mutant variants have increased expression of a nucleic acid sequence encoding a MYB-TF polypeptide. In a particular embodiment, plants carrying such mutant variants have preferentially increased expression of a nucleic acid sequence encoding a MYB-TF polypeptide in the endosperm of a plant seed.

T-DNA activation and TILLING are examples of technologies that enable the generation of genetic modifications comprising increasing expression in a plant of a nucleic acid sequence encoding a MYB-TF polypeptide, or comprising preferentially increasing expression of a nucleic acid sequence encoding a MYB-TF polypeptide in the endosperm of a plant seed.

Homologous recombination allows introduction in a genome of a selected nucleic acid sequence at a defined selected position. Homologous recombination is a standard technology used routinely in biological sciences for lower organisms such as yeast or the moss Physcomitrella. Methods for performing homologous recombination in plants have been described not only for model plants (Offring a et al. (1990) EMBO J. 9(10): 3077-84) but also for crop plants, for example rice (Terada et al. (2002) Nat Biotech 20(10): 1030-4; lida and Terada (2004) Curr Opin Biotech 15(2): 132-8). The nucleic acid sequence to be targeted is preferably the region controlling the natural expression of a nucleic acid sequence encoding a MYB-TF polypeptide in a plant. A strong constitutive promoter or alternatively, a strong endosperm-specific promoter, is introduced into this region, replacing it partly or substantially all of it.

A preferred method for introducing a genetic modification (which in this case need not be in the locus of a MYB-TF gene) is to introduce and express in a plant a nucleic acid sequence encoding a MYB-TF polypeptide, as defined hereinabove. The expression of the nucleic acid encoding a MYB-TF polypeptide is increased. In a particular embodiment, a preferred method for introducing a genetic modification (which in this case need not be in the locus of a MYB-TF gene) is to introduce and preferentially increase expression of a nucleic acid sequence encoding a MYB-TF polypeptide, as defined hereinabove, in the endosperm of a plant seed. The nucleic acid sequence to be introduced into a plant may be a full-length nucleic acid sequence or may be a portion or a hybridising sequence or another nucleic acid variant as hereinbefore defined.

The methods of the invention rely on increasing expression in a plant of a nucleic acid sequence encoding MYB-TF polypeptide. In a particular embodiment, the methods of the invention rely on preferentially increased expression of a nucleic acid sequence encoding a MYB-TF polypeptide, in the endosperm of a plant seed. Methods for increasing expression of genes or gene products are well documented in the art and include, for example, overexpression driven by appropriate promoters, the use of transcription enhancers or translation enhancers. Isolated nucleic acid sequences which serve as promoter or enhancer elements may be introduced in an appropriate position (typically upstream) of a non-heterologous form of a polynucleotide so as to upregulate expression of a nucleic acid sequence encoding a MYB-TF polypeptide. For example, endogenous promoters may be altered in vivo by mutation, deletion, and/or substitution (see, Kmiec, U.S. Pat. No. 5,565,350; Zarling et al., PCT/US93/03868), or isolated promoters may be introduced into a plant cell in the proper orientation and distance from a gene of the present invention so as to control the expression of the gene.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added may be derived from, for example, the nopaline synthase or octopine synthase genes, or alternatively from another plant gene, or less preferably from any other eukaryotic gene.

An intron sequence may also be added to the 5′ untranslated region (UTR) or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg (1988) Mol. Cell biol. 8: 4395-4405; Callis et al. (1987) Genes Dev 1:1183-1200). Such intron enhancement of gene expression is typically greatest when placed near the 5′ end of the transcription unit. Use of the maize introns Adh1-S intron 1, 2, and 6, the Bronze-1 intron are known in the art. For general information see: The Maize Handbook, Chapter 116, Freeling and Walbot, Eds., Springer, N.Y. (1994).

Other control sequences (besides promoter, enhancer, silencer, intron sequences, 3′UTR and/or 5′UTR regions) may be protein and/or RNA stabilizing elements.

The invention also provides genetic constructs and vectors to facilitate introduction and/or preferential expression of the nucleic acid sequences useful in the methods according to the invention, in the endosperm of a plant seed.

Therefore, there is provided a construct comprising:
(i) A nucleic acid sequence encoding a MYB-TF polypeptide as defined hereinabove;
(ii) One or more control sequences capable of driving expression in a plant, plant part or plant cell, of the nucleic acid sequence of (a); and optionally
(iii) A transcription termination sequence.

Preferably, one of the control sequences is at least one of: (i) a constitutive promoter, preferably a GOS2 promoter; or (ii) an endosperm-specific promoter, preferably a prolamin promoter.

Constructs useful in the methods according to the present invention may be constructed using recombinant DNA technology well known to persons skilled in the art. The gene constructs may be inserted into vectors, which may be commercially available, suitable for transforming into plants and suitable for expression of the gene of interest in the transformed cells. The invention therefore provides use of a gene construct as defined hereinabove in the methods of the invention.

Plants are transformed with a vector comprising the sequence of interest (i.e., a nucleic acid sequence encoding a MYB-TF polypeptide). The skilled artisan is well aware of the genetic elements that must be present on the vector in order to successfully transform, select and propagate host cells containing the sequence of interest. The sequence of interest is operably linked to one or more control sequences (at least to a promoter). The terms “regulatory element”, “control sequence” and “promoter” are all used interchangeably herein and are to be taken in a broad context to refer to regulatory nucleic acid sequences capable of effecting expression of the sequences to which they are ligated. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative that confers, activates or enhances expression of a nucleic acid molecule in a cell, tissue or organ. The term “operably linked” as used herein refers to a functional linkage between the promoter sequence and the gene of interest, such that the promoter sequence is able to initiate transcription of the gene of interest.

The term “constitutive promoter” as used herein refers to a promoter that is transcriptionally active during most, but not necessarily all, phases of growth and development and under most environmental conditions, in at least one cell, tissue or organ (such as the endosperm of a plant seed). Table 2 below gives examples of constitutive promoters.

A constitutive promoter is particularly useful in the methods of the invention. It should be clear that the applicability of the present invention is not restricted to the nucleic acid sequence encoding a MYB-TF polypeptide, as represented by SEQ ID NO: 1, nor is the applicability of the invention restricted to expression of a nucleic acid sequence encoding a MYB-TF polypeptide, when driven by a constitutive promoter.

TABLE 2
Examples of constitutive promoters

| Gene Source | Expression Pattern | Reference |
|---|---|---|
| Actin | Constitutive | McElroy et al., Plant Cell, 2: 163-171, 1990 |
| CAMV 35S | Constitutive | Odell et al., Nature, 313: 810-812, 1985 |
| CaMV 19S | Constitutive | Nilsson et al., Physiol. Plant. 100: 456-462, 1997 |
| GOS2 | Constitutive | de Pater et al., Plant J Nov; 2(6): 837-44, 1992 |
| Ubiquitin | Constitutive | Christensen et al., Plant Mol. Biol. 18: 675-689, |
| | | 1992 |
| Rice cyclophilin | Constitutive | Buchholz et al., Plant Mol Biol. 25(5): 837-43, |
| | | 1994 |
| Maize H3 histone | Constitutive | Lepetit et al., Mol. Gen. Genet. 231: 276-285, |
| | | 1992 |
| Actin 2 | Constitutive | An et al., Plant J. 10(1); 107-121, 1996 |

An organ-specific or tissue-specific promoter is one that is capable of preferentially initiating transcription in certain organs or tissues, such as the leaves, roots, seed tissue etc. For example, a “root-specific promoter” is a promoter that is transcriptionally active predominantly in plant roots, substantially to the exclusion of any other parts of a plant, whilst still allowing for any leaky expression in these other plant parts.

An endosperm-specific promoter refers to any promoter able to preferentially drive expression of the gene of interest in the endosperm of a plant seed. Reference herein to “preferentially” driving expression in the endosperm of a plant seed is taken to mean driving expression of any sequence operably linked thereto in the endosperm substantially to the exclusion of driving expression elsewhere in the plant, apart from any residual expression due to leaky promoter expression. For example, the prolamin promoter shows strong expression in the endosperm of a plant seed, with leakiness in meristem, more specifically the shoot meristem and/or discrimination centre in the meristem. The endosperm-specific promoter may be either a natural or a synthetic promoter.

Preferably, the endosperm-specific promoter is a promoter isolated from a prolamin gene, such as a rice prolamin RP6 promoter (Wen et al., (1993) Plant Physiol 101(3): 1115-6) as represented by SEQ ID NO: 29 or by SEQ ID NO: 41, or a promoter of similar strength and/or a promoter with a similar expression pattern as the rice prolamin promoter. Similar strength and/or similar expression pattern may be analysed, for example, by coupling the promoters to a reporter gene and checking the function of the reporter gene in tissues of the plant. One well-known reporter gene is beta-glucuronidase and the calorimetric GUS stain used to visualize beta-glucuronidase activity in plant tissue. It should be clear that the applicability of the present invention is not restricted to the nucleic acid sequence encoding a MYB-TF polypeptide represented by SEQ ID NO: 2, nor is the applicability of the invention restricted to expression of a nucleic acid encoding a MYB-TF polypeptide when driven by a prolamin promoter. Examples of other endosperm-specific promoters which may also be used perform the methods of the invention are shown in Table 3 below.

TABLE 3
Examples of endosperm-specific promoters for use in the present invention

| Gene source | Reference |
|---|---|
| glutelin (rice) | Takaiwa et al. (1986) Mol Gen Genet 208: 15-22 |
| | Takaiwa et al. (1987) FEBS Letts. 221: 43-47 |
| zein | Matzke et al., (1990) Plant Mol Biol 14(3): 323-32 |
| wheat LMW and HMW glutenin-1 | Colot et al. (1989) Mol Gen Genet 216: 81-90 |
| | Anderson et al. (1989) NAR 17: 461-2 |
| wheat SPA | Albani et al. (1997) Plant Cell 9: 171-184 |
| wheat gliadins | Rafalski et al. (1984) EMBO 3: 1409-15 |
| barley ltr1 promoter | Diaz et al. (1995) Mol Gen Genet 248(5): 592-8 |
| barley B1, C, D, hordein | Cho et al. (1999) Theor Appl Genet 98: 1253-62 |
| | Muller et al. (1993) Plant J 4: 343-55 |
| | Sorenson et al. (1996) Mol Gen Genet 250: 750-60 |
| barley DOF | Mena et al, (1998) Plant J 116(1): 53-62 |
| blz2 | Onate et al. (1999) J Biol Chem 274(14): 9175-82 |
| synthetic promoter | Vicente-Carbajosa et al. (1998) Plant J 13: 629-640 |
| rice prolamin NRP33 | Wu et al, (1998) Plant Cell Physiol 39(8) 885-889 |
| rice globulin Glb-1 | Wu et al. (1998) Plant Cell Physiol 39(8) 885-889 |
| rice globulin REB/OHP-1 | Nakase et al. (1997) Plant Molec Biol 33: 513-522 |
| rice ADP-glucose PP | Russell et al. (1997) Trans Res 6: 157-68 |
| maize ESR gene family | Opsahl-Ferstad et al. (1997) Plant J 12: 235-46 |
| sorgum kafirin | DeRose et al. (1996) Plant Molec Biol 32: 1029-35 |

Another example of a tissue-specific promoter is one that is capable of preferentially initiating transcription in young expanding tissues, such the beta-expansin promoter. Preferably, the beta-expansin promoter is from rice, more preferably substantially similar to SEQ ID NO: 40, most preferably is as represented by SEQ ID NO: 40. Such a promoter is also useful in performing the methods according to the invention.

Optionally, one or more terminator sequences (also a control sequence) may be used in the construct introduced into a plant. The term “terminator” encompasses a control sequence which is a DNA sequence at the end of a transcriptional unit which signals 3′ processing and polyadenylation of a primary transcript and termination of transcription. Additional regulatory elements may include transcriptional as well as translational enhancers. Those skilled in the art will be aware of terminator and enhancer sequences that may be suitable for use in performing the invention. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

The genetic construct may optionally comprise a selectable marker gene. As used herein, the term “selectable marker gene” includes any gene that confers a phenotype on a cell in which it is expressed to facilitate the identification and/or selection of cells that are transfected or transformed with a nucleic acid construct of the invention. Suitable markers may be selected from markers that confer antibiotic or herbicide resistance, that introduce a new metabolic trait or that allow visual selection. Examples of selectable marker genes include genes conferring resistance to antibiotics (such as nptII that phosphorylates neomycin and kanamycin, or hpt, phosphorylating hygromycin, or genes conferring resistance to, for example, bleomycin, streptomycin, tetracyclin, chloramphenicol, ampicillin, gentamycin, geneticin (G418), spectinomycin or blasticidin), to herbicides (for example bar which provides resistance to Basta®; aroA or gox providing resistance against glyphosate, or the genes conferring resistance to, for example, imidazolinone, phosphinothricin or sulfonylurea), or genes that provide a metabolic trait (such as manA that allows plants to use mannose as sole carbon source or xylose isomerase for the utilisation of xylose, or antinutritive markers such as the resistance to 2-deoxyglucose). Expression of visual marker genes results in the formation of colour (for example β-glucuronidase, GUS or β-galactosidase with its coloured substrates, for example X-Gal), luminescence (such as the luciferin/luceferase system) or fluorescence (Green Fluorescent Protein, GFP, and derivatives thereof). This list represents only a small number of possible markers. The skilled worker is familiar with such markers. Different markers are preferred, depending on the organism and the selection method.

The present invention also encompasses plants obtainable by the methods according to the present invention. The present invention therefore provides plants, plant parts or plant cells thereof obtainable by the method according to the present invention, which plants or parts or cells thereof comprise an isolated nucleic acid encoding a MYB-TF polypeptide under the control of a constitutive promoter as defined herein above. In a particular embodiment, the present invention provides plants, plant parts or plant cells thereof obtainable by the method according to the present invention, which plants or parts or cells thereof comprise an isolated nucleic acid encoding a MYB-TF polypeptide under the control of an endosperm-specific promoter.

The invention also provides a method for the production of transgenic plants having increased yield relative to control plants, comprising introducting and expressing in a plant of a nucleic acid sequence encoding a MYB-TF polypeptide. In a particular embodiment, the invention provides a method for the production of transgenic plants having increased yield relative to control plants, comprising introducting and preferentially expressing of a nucleic acid sequence encoding a MYB-TF polypeptide in the endosperm of a plant seed.

More specifically, the present invention provides a method for the production of transgenic plants having increased yield relative to control plants, which method comprises:
(i) introducing and expressing in a plant, plant part or a plant cell a nucleic acid sequence encoding a MYB-TF polypeptide; and
(ii) cultivating the plant cell under conditions promoting plant growth and development and wherein the expression of the nucleic acid sequence encoding a MYB-TF polypeptide is driven by a constitutive promoter, or by an endosperm-specific promoter.

The nucleic acid sequence may be introduced directly into a plant cell or into the plant itself (including introduction into a tissue, organ or any other part of a plant). According to a preferred feature of the present invention, the nucleic acid sequence is preferably introduced into a plant by transformation.

The term “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated from there. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

Transformation of plant species is now a fairly routine technique. Advantageously, any of several transformation methods may be used to introduce the gene of interest into a suitable ancestor cell. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al. (1982) Nature 296, 72-74; Negrutiu I et al. (1987) Plant Mol Biol 8: 363-373); electroporation of protoplasts (Shillito R. D. et al. (1985) Bio/Technol 3, 1099-1102); microinjection into plant material (Crossway A et al. (1986) Mol. Gen Genet 202: 179-185); DNA or RNA-coated particle bombardment (Klein T M et al. (1987) Nature 327: 70) infection with (non-integrative) viruses and the like. Transgenic rice plants with increased expression a gene/nucleic acid sequence encoding a MYB-TF polypeptide are preferably produced via Agrobacterium-mediated transformation using any of the well known methods for rice transformation, such as described in any of the following: published European patent application EP 1198985 A1, Aldemita and Hodges (Planta 199: 612-617, 1996); Chan et al. (Plant Mol Biol 22 (3): 491-506, 1993), Hiei et al. (Plant J 6 (2): 271-282, 1994), which disclosures are incorporated by reference herein as if fully set forth. In the case of corn transformation, the preferred method is as described in either Ishida et al. (Nat. Biotechnol 14(6): 745-50, 1996) or Frame et al. (Plant Physiol 129(1): 13-22, 2002), which disclosures are incorporated by reference herein as if fully set forth.

Generally after transformation, plant cells or cell groupings are selected for the presence of one or more markers which are encoded by plant-expressible genes co-transferred with the gene of interest, following which the transformed material is regenerated into a whole plant.

Following DNA transfer and regeneration, putatively transformed plants may be evaluated, for instance using Southern analysis, for the presence of the gene of interest, copy number and/or genomic organisation. Alternatively or additionally, expression levels of the newly introduced DNA may be monitored using Northern and/or Western analysis, or quantitative PCR, all techniques being well known to persons having ordinary skill in the art.

The generated transformed plants may be propagated by a variety of means, such as by clonal propagation or classical breeding techniques. For example, a first generation (or T1) transformed plant may be selfed to give homozygous second generation (or T2) transformants, and the T2 plants further propagated through classical breeding techniques.

The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. More specifically, the present invention provides for plants, plant parts, or plant cells obtainable by the methods of the invention, wherein said plants, or parts or cells thereof, comprises an isolated nucleic acid encoding a MYB-TF polypeptide, which nucleic acid is operably linked to a constitutive promoter, or to an endosperm-specific promoter.

The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also includes host cells containing an isolated nucleic acid sequence encoding a MYB-TF polypeptide. Preferred host cells according to the invention are plant cells.

The invention also extends to harvestable parts of a plant such as, but not limited to seeds, leaves, fruits, flowers, stems, rhizomes, tubers and bulbs. The invention furthermore relates to products derived, preferably directly derived, from a harvestable part of such a plant, such as dry pellets or powders, oil, fat and fatty acids, starch or proteins.

The present invention also encompasses use of nucleic acid sequences encoding MYB-TF polypeptides and use of MYB-TF polypeptides in increasing plant yield as defined hereinabove in the methods of the invention.

Nucleic acid sequences encoding MYB-TF polypeptides, or MYB-TF polypeptides, may find use in breeding programmes in which a DNA marker is identified which may be genetically linked to a MYB-TF gene. The genes/nucleic acid sequences, or the MYB-TF polypeptides may be used to define a molecular marker. This DNA or protein marker may then be used in breeding programmes to select plants having increased yield as defined hereinabove in the methods of the invention.

Allelic variants of a MYB-TF gene/nucleic acid sequence may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased yield. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

A nucleic acid sequence encoding a MYB-TF polypeptide may also be used as probes for genetically and physically mapping the genes that they are a part of, and as markers for traits linked to those genes. Such information may be useful in plant breeding in order to develop lines with desired phenotypes. Such use of MYB-TF nucleic acid sequences requires only a nucleic acid sequence of at least 15 nucleotides in length. The MYB-TF nucleic acid sequences may be used as restriction fragment length polymorphism (RFLP) markers. Southern blots (Sambrook J, Fritsch EF and Maniatis T (1989) Molecular Cloning, A Laboratory Manual) of restriction-digested plant genomic DNA may be probed with the MYB-TF nucleic acid sequences. The resulting banding patterns may then be subjected to genetic analyses using computer programs such as MapMaker (Lander et al. (1987) Genomics 1: 174-181) in order to construct a genetic map. In addition, the nucleic acid sequences may be used to probe Southern blots containing restriction endonuclease-treated genomic DNAs of a set of individuals representing parent and progeny of a defined genetic cross. Segregation of the DNA polymorphisms is noted and used to calculate the position of the MYB-TF nucleic acid sequence in the genetic map previously obtained using this population (Botstein et al. (1980) Am. J. Hum. Genet. 32:314-331).

The production and use of plant gene-derived probes for use in genetic mapping is described in Bernatzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridisation (FISH) mapping (Trask (1991) Trends Genet. 7:149-154). Although current methods of FISH mapping favor use of large clones (several kb to several hundred kb; see Laan et al. (1995) Genome Res. 5:13-20), improvements in sensitivity may allow performance of FISH mapping using shorter probes.

A variety of nucleic acid amplification-based methods for genetic and physical mapping may be carried out using the nucleic acid sequences. Examples include allele-specific amplification (Kazazian (1989) J. Lab. Clin. Med 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

The methods according to the present invention result in plants having increased yield, as described hereinbefore. This increased yield may also be combined with other economically advantageous traits, such as further yield-enhancing traits, tolerance to other abiotic and biotic stresses, traits modifying various architectural features and/or biochemical and/or physiological features.

### DESCRIPTION OF FIGURES

The present invention will now be described with reference to the following figures in which:

FIG. 1 represents the schematic structure a MYB-TF polypeptide comprising from N-terminus to C-terminus an R2R3 MYB DNA binding domain (large circle) comprising two MYB repeats (two smaller circles), and a MYB4 domain (boxed). The regions of the polypeptide outside of these domains are variable regions.

FIG. 2 represents a table of sequences related to a MYB-TF polypeptide, as tentatively assembled by The Institute for Genomic Research (TIGR) (TC924083). The Eukaryotic Gene Orthologs (EGO) database may be used to identify such related sequences, either by keyword search or by using the BLAST algorithm with the nucleic acid or polypeptide sequence of interest. This list is not exhaustive, more related sequences may be identified in the Entrez Nucleotides database at the National Center for Biotechnology Information (NCBI) using database sequence search tools.

FIG. 3 is a phylogenetic tree of plant R2R3 MYB-TF polypeptides, generated by Kranz et al. (1998) Plant Journal 16(2): 263-276. The region comprising the clade of interest has been expanded from the original tree. SEQ ID NO: 2 (from Otyza sativa) and SEQ ID NO: 4 (from Arabidopsis thaliana) are indicated and their position in the tree marked with a bold arrow. The origin of the clade of interest is marked with a circle.

FIG. 4 is an alignment of MYB-TF polypeptide sequences. The sequences were aligned using AlignX program from Vector NTI suite (InforMax, Bethesda, Md.). Multiple alignment was done with a gap opening penalty of 10 and a gap extension of 0.01. Minor manual editing was also carried out where necessary to better position some conserved regions. The line shown indicates the separation of MYB-TF polypeptide sequences useful in performing the methods of the invention, and other MYB-TF polypeptide sequences. The two MYB repeats (R2 and R3) are heavily boxed across all sequences shown. The 3 helices within these two repeats are lightly boxed. The conserved Trp (W) residues are marked with an arrow. The MYB4 domain at the C-terminus of the aligned polypeptide sequences is heavily boxed but only across the MYB-TF polypeptide sequences useful in performing the methods of the invention.

FIG. 5 shows a vector for expression in Oryza sativa of an Oryza sativa nucleic acid sequence encoding a MYB-TF polypeptide, under the control of a GOS2 promoter (pGOS2) or of a prolamin promoter (pProl) or of a beta-expansin promoter (pExp).

FIG. 6 details examples of sequences useful in performing the methods according to the present invention.

### EXAMPLES

The present invention will now be described with reference to the following examples, which are by way of illustration alone. The following examples are not intended to completely define or otherwise limit the scope of the invention.

### Example 1

### Identification of Sequences Related to the Nucleic Acid Sequence Used in the Methods of the Invention

Sequences (full length cDNA, ESTs or genomic) related to the nucleic acid sequence used in the methods of the present invention were identified amongst those maintained in the Entrez Nucleotides database at the National Center for Biotechnology Information (NCBI)(http://www.ncbi.nlm.nih.gov) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid or polypeptide sequences to sequence databases and by calculating the statistical significance of matches. For example, the polypeptide encoded by the nucleic acid sequence of the present invention was used for the TBLASTN algorithm, with default settings and the filter to ignore low complexity sequences set off. The output of the analysis was viewed by pairwise comparison, and ranked according to the probability score (E-value), where the score reflects the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid (or polypeptide) sequences over a particular length. In some instances, the default parameters may be adjusted to modify the stringency of the search. For example the E-value may be increased to show less stringent matches. This way, short nearly exact matches may be identified.

The Table below provides a list of nucleic acid sequences related to the nucleic acid sequence used in the methods of the present invention.

TABLE A
nucleic acid sequences related to the nucleic acid sequence used in the methods of
the present invention

| | Nucleic | | | | |
|---|---|---|---|---|---|
| | acid SEQ | Polypeptide | | NCBI accession | Source |
| Name | ID NO | SEQ ID NO | | number | organism |
| Orysa_MYB4 | 1 | 2 | full | D88620 | Oryza sativa |
| | | | length | | |
| Arath_MYB5 | 3 | 4 | full | NM_112200 | Arabidopsis |
| | | | length | | thaliana |
| Goshi_BNLGHi233 | 5 | 6 | full | AF336278 | Gossypium |
| | | | length | | hirsutum |
| Lotco_MYB4 | 7 | 8 | full | AP007766.1 | Lotus |
| | | | length | | corniculatus |
| Medtr_MYB4 | 9 | 10 | full | AC146760.13 | Medicago |
| | | | length | | trunculata |
| Pethy_MYB4 | 11 | 12 | full | AY97332 | Petunia |
| | | | length | | hybrida |
| Poptr_MYB4 | 13 | 14 | full | CA825038 CN520600 | Populus |
| | | | length | | tremuloides |
| Vitvi_MYB5b | 15 | 16 | full | AY899404 | Vitis vinifera |
| | | | length | | |
| Zeama_MYB4 | 17 | 18 | full | CZ363319 AF470078 | Zea mays |
| | | | length | | |
| Antma_MYB4 | 19 | 20 | partial | AJ568077 | Antirrhinum |
| | | | | | majus |
| Lyces_MYB4 | 21 | 22 | partial | DU949128.1 | Lycopersicon |
| | | | | | esculentum |
| Solch_MYB4 | 23 | 24 | partial | DN979550 | Solanum |
| | | | | | chacoense |
| Triae_MYB4 | 25 | 26 | partial | CD897518 | Triticum |
| | | | | | aestivum |
| Brana_MYB4 | 32 | 33 | full | BN06MC21765_47591244 | Brassica |
| | | | length | | napus |
| Eucgr_MYB4 | 34 | 35 | Full | ADW16600 | Eucalyptus |
| | | | length | | grandis |
| Maldo_MYB4 | 36 | 37 | full | EG631283 | Malus |
| | | | length | | domestica |
| Vitvi_MYB4 | 38 | 39 | full | AY555190 | Vitis vinifera |
| Mybcs1 | | | length | | |

In some instances, related sequences have tentatively been assembled and publicly disclosed by research institutions, such as The Institute for Genomic Research (TIGR). The Eukaryotic Gene Orthologs (EGO) database may be used to identify such related sequences, either by keyword search or by using the BLAST algorithm with the nucleic acid or polypeptide sequence of interest. The output of such a search may be seen in FIG. 2.

### Example 2

### Alignment of Relevant Polypeptide Sequences

AlignX from the Vector NTI (Invitrogen) is based on the popular Clustal algorithm of progressive alignment (Thompson et al. (1997) Nucleic Acids Res 25:4876-4882; Chenna et al. (2003). Nucleic Acids Res 31:3497-3500). A phylogenetic tree can be constructed using a neighbour-joining clustering algorithm. Default values are for the gap open penalty of 10, for the gap extension penalty of 0, 1 and the selected weight matrix is Blosum 62 (if polypeptides are aligned).

The result of the multiple sequence alignment using polypeptides relevant in identifying the ones useful in performing the methods of the invention is shown in FIG. 4. The sequences were aligned using AlignX program from Vector NTI suite (InforMax, Bethesda, Md.). Multiple sequence alignment was done with a gap opening penalty of 10 and a gap extension of 0.01. Minor manual editing was also carried out where necessary to better position some conserved regions.

The line shown indicates the separation of MYB-TF polypeptide sequences useful in performing the methods of the invention, and other MYB-TF polypeptide sequences. The two MYB repeats (R2 and R3) are heavily boxed across all sequences shown. The 3 helices within these two repeats are lightly boxed. The conserved Trp (W) residues are marked with an arrow. The MYB4 domain at the C-terminus of the aligned polypeptide sequences is heavily boxed but only for the MYB-TF polypeptide sequences that are useful in performing the methods of the invention.

### Example 3

### Calculation of Global Percentage Identity Between Polypeptide Sequences Useful in Performing the Methods of the Invention

Global percentages of similarity and identity between full length polypeptide sequences useful in performing the methods of the invention were determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line.

Parameters used in the comparison were:
Scoring matrix: Blosum62
First Gap: 12
Extending gap: 2

Results of the software analysis are shown in Table B for the global similarity and identity over the full length of the polypeptide sequences (excluding the partial polypeptide sequences). Percentage identity is given above the diagonal and percentage similarity is given below the diagonal.

The percentage identity between the polypeptide sequences useful in performing the methods of the invention ranges between 40% and 56% amino acid identity compared to SEQ ID NO: 2.

TABLE B
MatGAT results for global similarity and identity over the full length of the MYB-TF
polypeptide sequences useful in performing the methods of the invention.

| | 1 | 2 | 3 | 4 | 5 | 6 | 7 | 8 | 9 | 10 | 11 | 12 |
|---|---|---|---|---|---|---|---|---|---|---|---|---|
| 1. Orysa_MYB4 | | 41.2 | 45.1 | 44.2 | 45.1 | 47.8 | 40.1 | 43 | 42.5 | 41.1 | 42.7 | 55.9 |
| 2. Maldo_MYB4 | 50.1 | | 41.8 | 43.4 | 53.9 | 47.6 | 50.7 | 50.1 | 52.1 | 50.4 | 56.6 | 39 |
| 3. Arath_MYB5 | 59.6 | 48.2 | | 78.9 | 48.8 | 54.1 | 43.1 | 50.5 | 45.4 | 47.1 | 47.3 | 44.7 |
| 4. Brana_MYB4 | 57.7 | 50.1 | 84.9 | | 48.2 | 52.5 | 44.8 | 49.6 | 45.7 | 46.4 | 47.3 | 42.6 |
| 5. Eucgr_MYB4 | 57.2 | 62.7 | 56.9 | 58.9 | | 56.5 | 53.5 | 56.5 | 58.3 | 55 | 58.1 | 44.9 |
| 6. Goshi_BNLGHi233 | 59.6 | 54.9 | 65.9 | 64.5 | 66.2 | | 50 | 58.7 | 54.6 | 52.2 | 54.8 | 47.3 |
| 7. Lotco_MYB4 | 50.3 | 61.6 | 50.6 | 53.9 | 61.5 | 56.7 | | 49.4 | 49.7 | 50 | 54.7 | 38.8 |
| 8. Pethy_MYB4 | 58 | 58.8 | 57.3 | 58 | 67.9 | 65.8 | 58.2 | | 52.9 | 49.2 | 55.1 | 44.3 |
| 9. Poptr_MYB4 | 53.2 | 61.3 | 55.7 | 56.6 | 66.8 | 58.9 | 61.2 | 62.7 | | 51.3 | 55.4 | 39.8 |
| 10. Vitvi_MYB4 MybCs1 | 50.6 | 61.1 | 55.3 | 55.3 | 65 | 58.8 | 62.4 | 61.3 | 63.1 | | 59 | 39.3 |
| 11. Vitvi_MYB5b | 54.3 | 68.9 | 54 | 57.6 | 70.7 | 62.4 | 67.6 | 65.9 | 68 | 72.2 | | 41.5 |
| 12. Zeama_MYB4 | 66.9 | 48.2 | 60.4 | 56.3 | 56.2 | 60 | 49.1 | 58.7 | 51.3 | 51.6 | 52.7 | |

The conserved MYB4 domain as represented by SEQ ID NO: 28 (DDDFSSFLDSLIND) is comprised in SEQ ID NO: 2 (amino acid coordinates 231-244; see FIG. 4). When the percentage identity is calculated between the conserved MYB4 domain of the polypeptide sequences useful in performing the methods of the invention and the conserved MYB4 domain as represented by SEQ ID NO: 28 (instead of between the full length polypeptides), then at least 60% amino acid identity is reached. For example, if five amino acid residues from a conserved MYB4 domain differ when aligned to the conserved MYB4 domain as represented by SEQ ID NO: 28, 64% amino acid identity is then calculated.

### Example 4

### Identification of Domains Comprised in Polypeptide Sequences Useful in Performing the Methods of the Invention

The Integrated Resource of Protein Families, Domains and Sites (InterPro) database is an integrated interface for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, ProDom and Pfam, Smart and TIGRFAMs, each database having its own search algorithms and entry accession numbers. Interpro is hosted at the European Bioinformatics Institute in the United Kingdom.

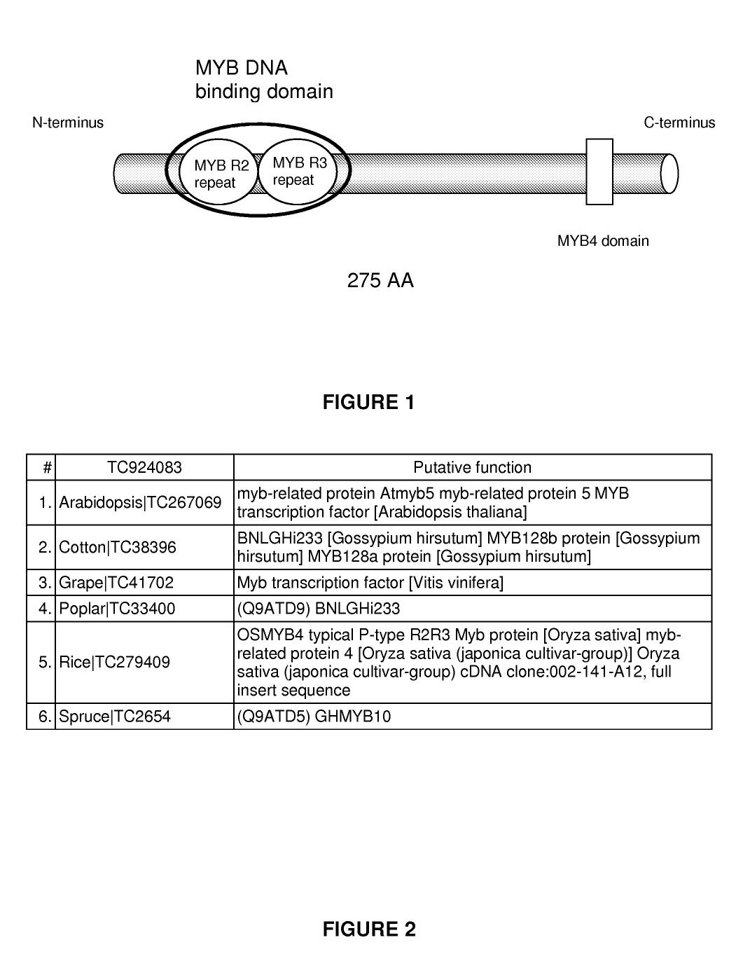

The results of the InterPro scan of the polypeptide sequence as represented by SEQ ID NO: 2 are presented in Table C and FIGS. 1 and 4.

TABLE C
InterPro scan results of the polypeptide sequence as represented by
SEQ ID NO: 2.

| InterPro | | | |
|---|---|---|---|
| accession | Originating | Original accession | |
| number | database | number | Accession name |
| IPR001005 | Pfam | PF00249 | Myb_DNA- |
| | | | binding |
| | SMART | SM00717 | SANT |
| | Profile | PS50090 | MYB_3 |
| | Prosite | PS00334 | MYB_2 |
| IPR009057 | SUPERFAMILY | SF46689 | Homeodomain- |
| | | | like |
| No IPR | Panther | PTHR10641 | MYB-related |
| integrated | | | |
| | Panther | PTHR10641:SF30 | R2R3-MYB |
| | | | transcription |
| | | | factor |

### Example 5

### Cloning of the Nucleic Acid Sequence Used in the Methods of the Invention

DNA manipulation: unless otherwise stated, recombinant DNA techniques are performed according to standard protocols described in (Sambrook (2001) Molecular Cloning: a laboratory manual, 3rd Edition Cold Spring Harbor Laboratory Press, CSH, New York) or in Volumes 1 and 2 of Ausubel et al. (1994), Current Protocols in Molecular Biology, Current Protocols. Standard materials and methods for plant molecular work are described in Plant Molecular Biology Labfase (1993) by R.D.D. Croy, published by BIOS Scientific Publications Ltd (UK) and Blackwell Scientific Publications (UK).

The nucleic acid sequence used in the methods of the invention was amplified by PCR using as template a custom-made Oryza sativa seedlings cDNA library (in pCMV Sport 6.0; Invitrogen, Paisley, UK). PCR was performed using Hifi Taq DNA polymerase in standard conditions, using 200 ng of template in a 50 μl PCR mix. The primers used were prm05233 (SEQ ID NO: 30; sense, start codon in bold, AttB1 site in italic: 5′-GGGGACAAGTTTGTACAAAAAAGCAGGCTTAAACAATGAAGCGGAAGCGG 3′)

and prm05234 (SEQ ID NO: 31; reverse, complementary, AttB2 site in italic: 5′ GGGGACCACTTTGTACAAGAAAGCTGGGTAATCTCCCGAAAGATTACTGT 3′),
which include the AttB sites for Gateway recombination. The amplified PCR fragment was purified also using standard methods. The first step of the Gateway procedure, the BP reaction, was then performed, during which the PCR fragment recombines in vivo with the pDONR201 plasmid to produce, according to the Gateway terminology, an “entry clone”, pMYB-TF. Plasmid pDONR201 was purchased from Invitrogen, as part of the Gateway® technology.

### Example 6

### Expression Vector Construction

The entry clone was subsequently used in an LR reaction with a destination vector used for Oryza sativa transformation. This vector contains as functional elements within the T-DNA borders: a plant selectable marker; a screenable marker expression cassette; and a Gateway cassette intended for LR in vivo recombination with the nucleic acid sequence of interest already cloned in the entry clone.

In one example, a rice GOS2 promoter (pGOS2; SEQ ID NO: 39) for constitutive expression was located upstream of this Gateway cassette.

In a second example, a rice prolamin promoter (pProl; SEQ ID NO: 29 or SEQ ID NO: 41) for endosperm-specific expression was located upstream of this Gateway cassette.

In a third example, a rice beta-expansin promoter (pExp; SEQ ID NO: 40) for expression in young expanding tissues was located opstream of this Gateway cassette.

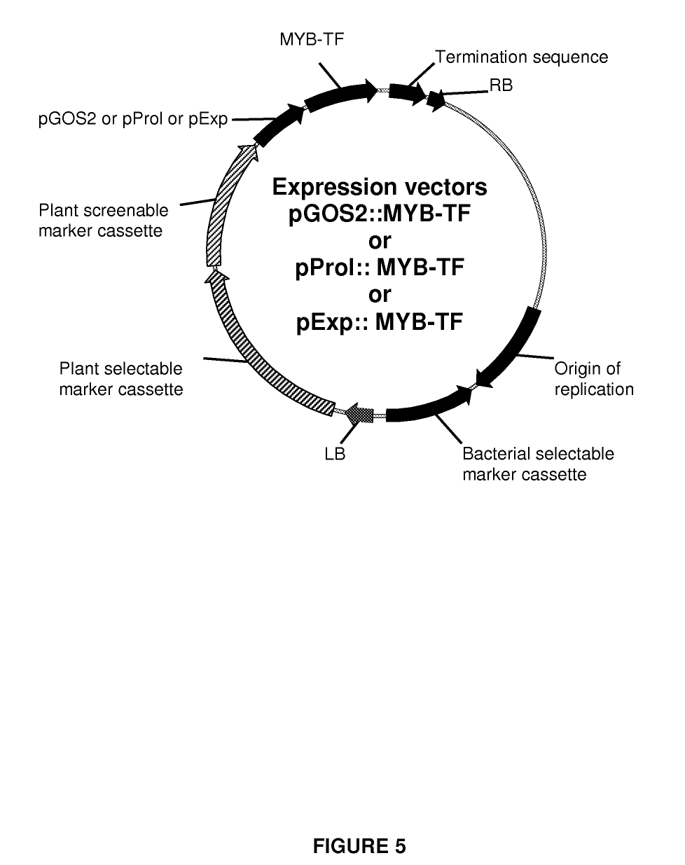

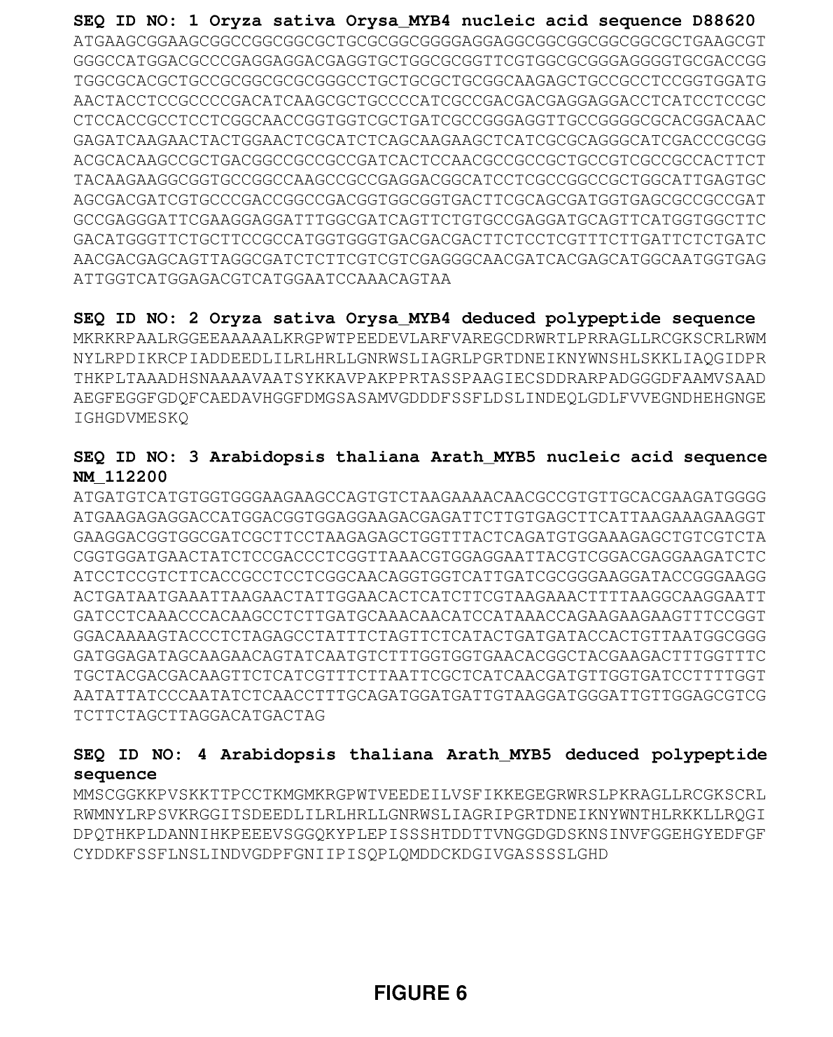

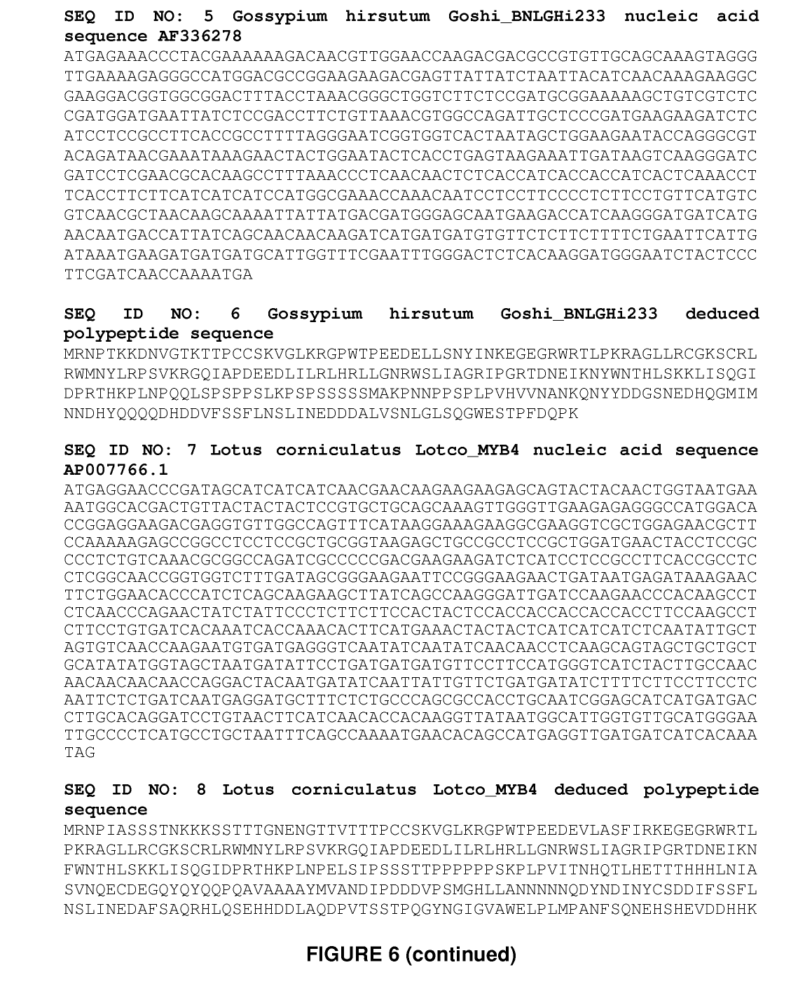

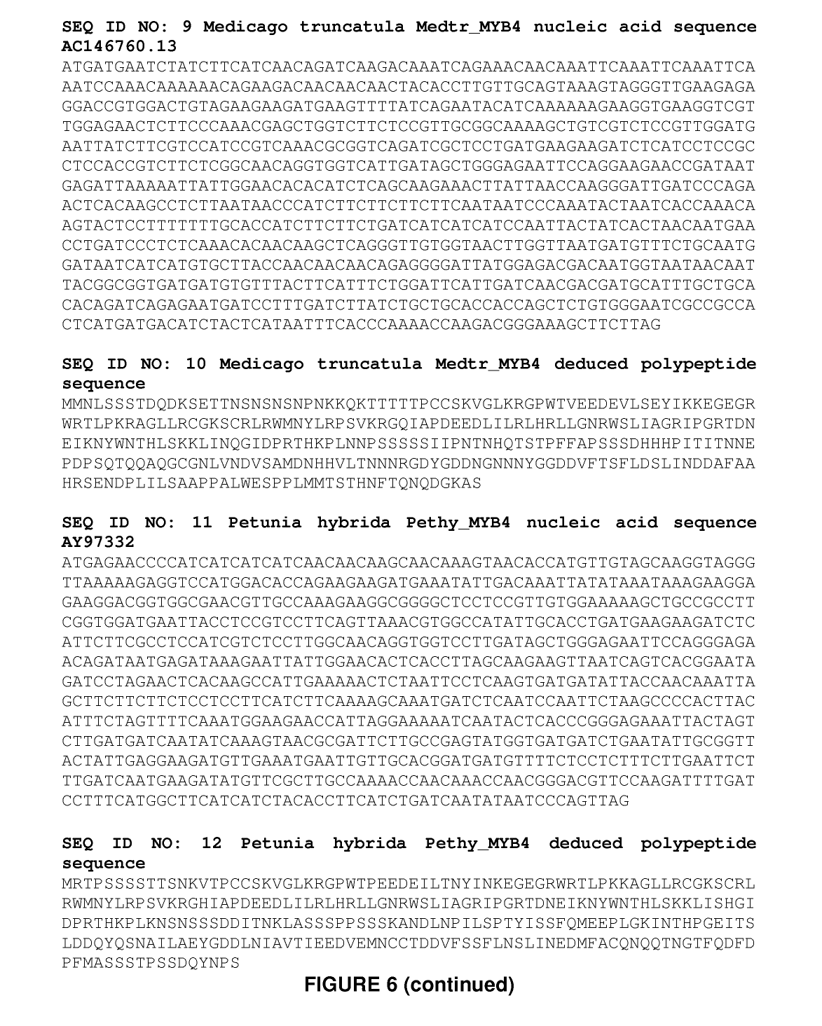

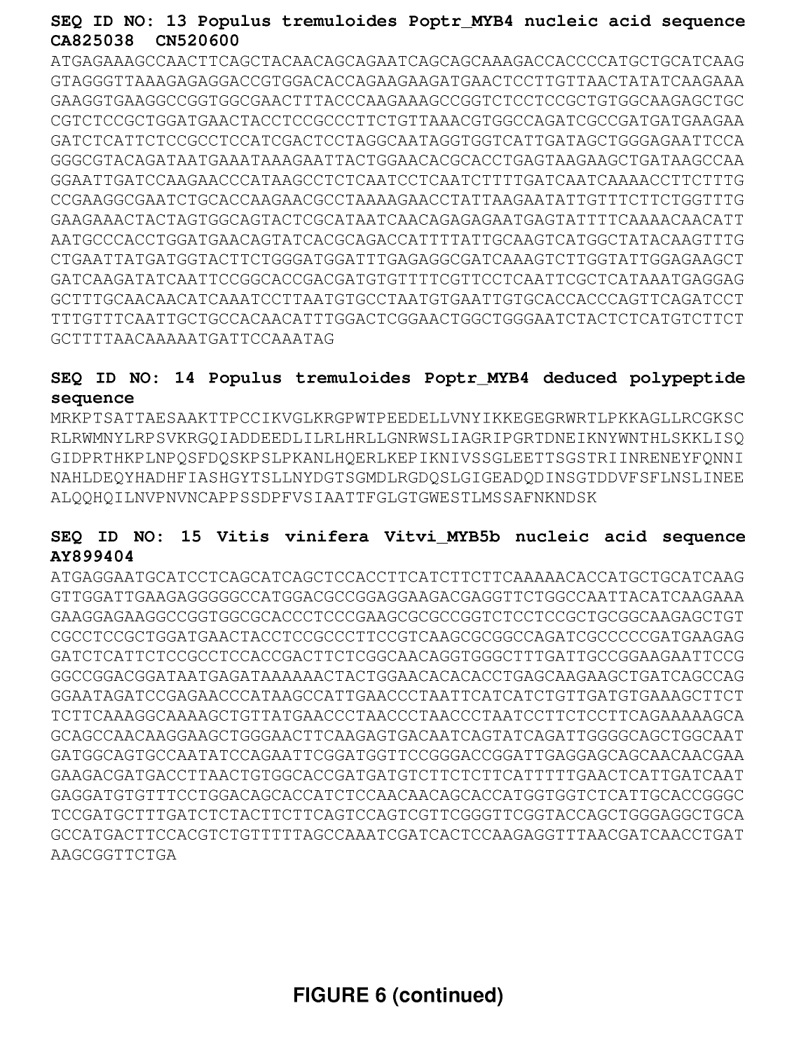

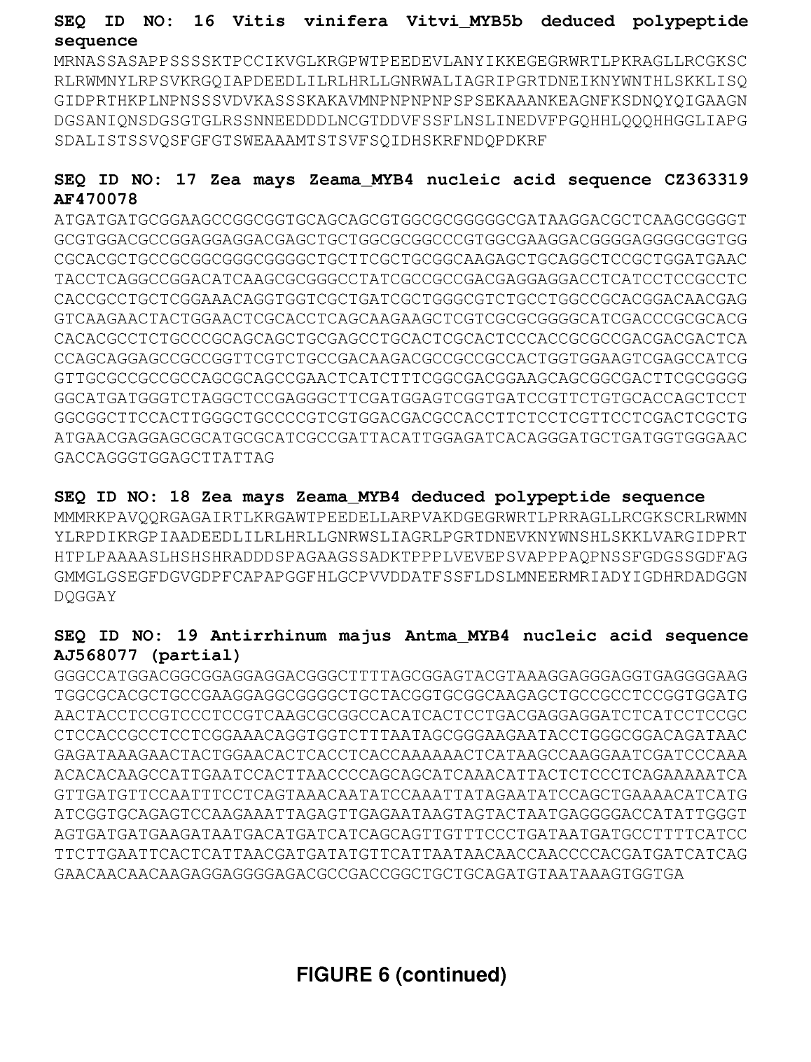

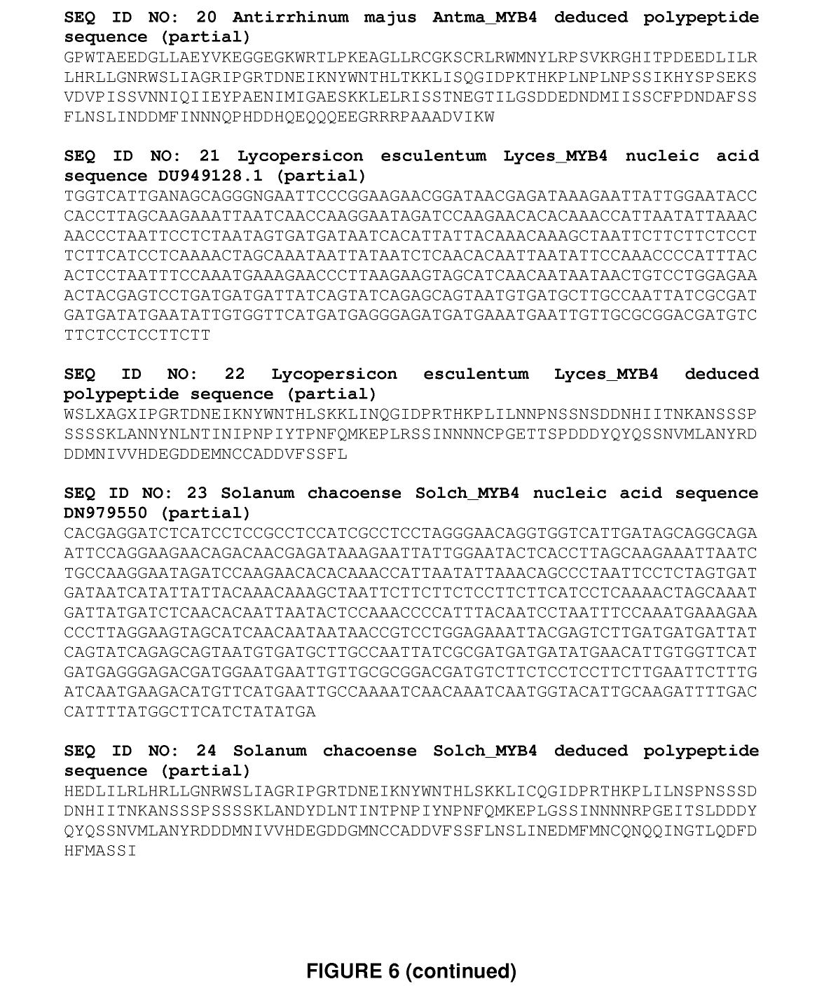

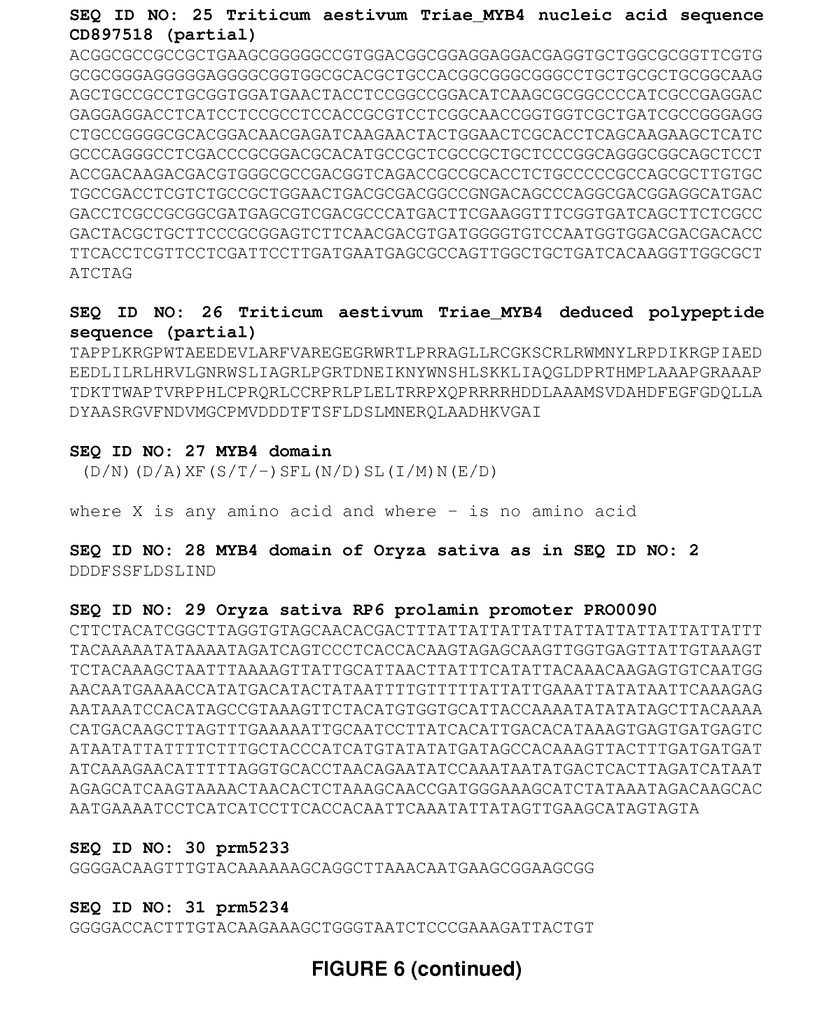

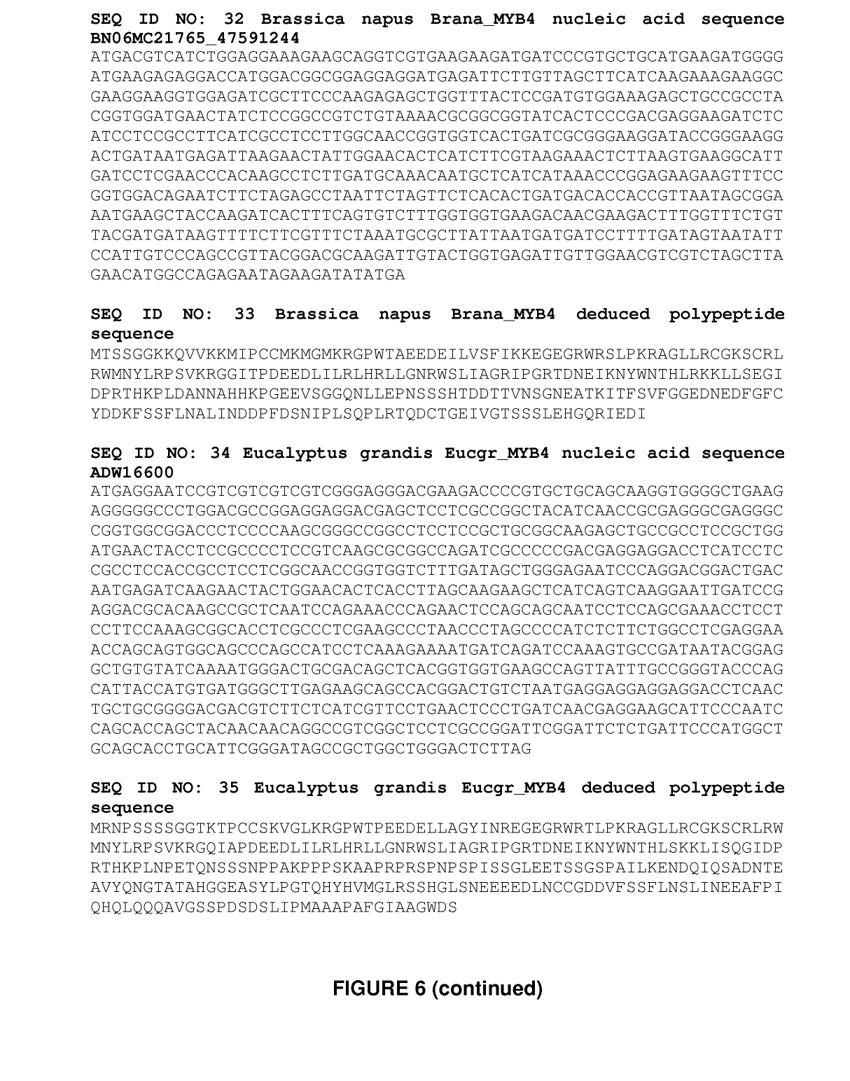

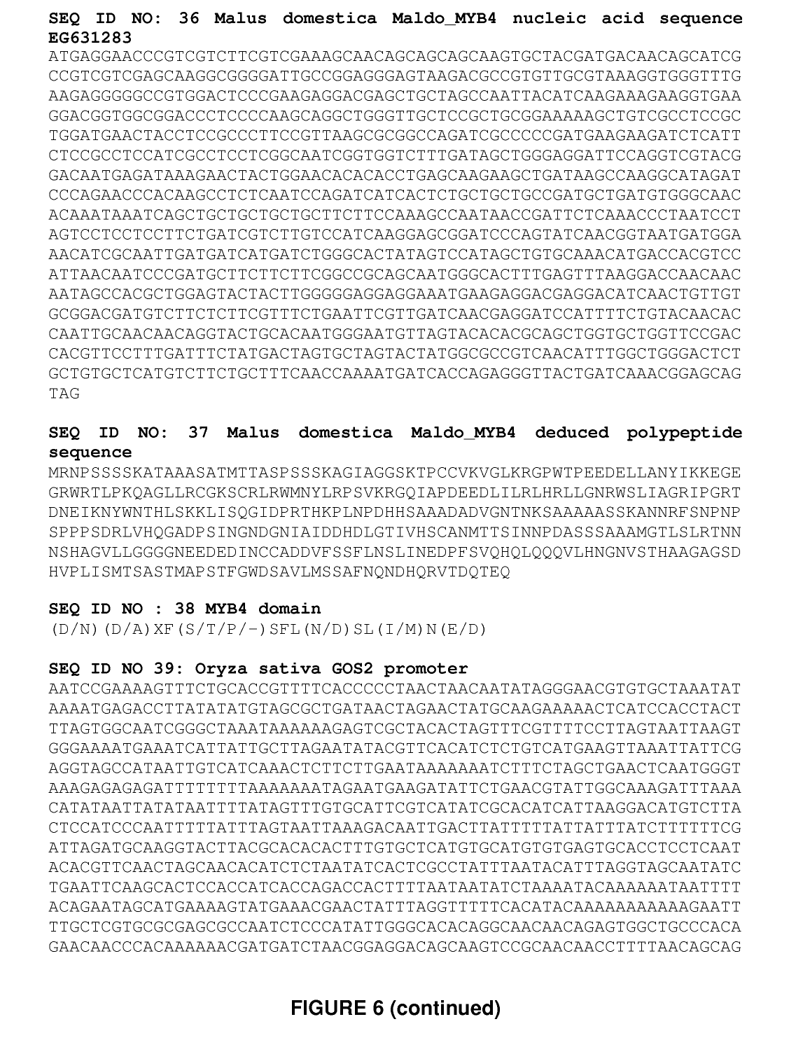

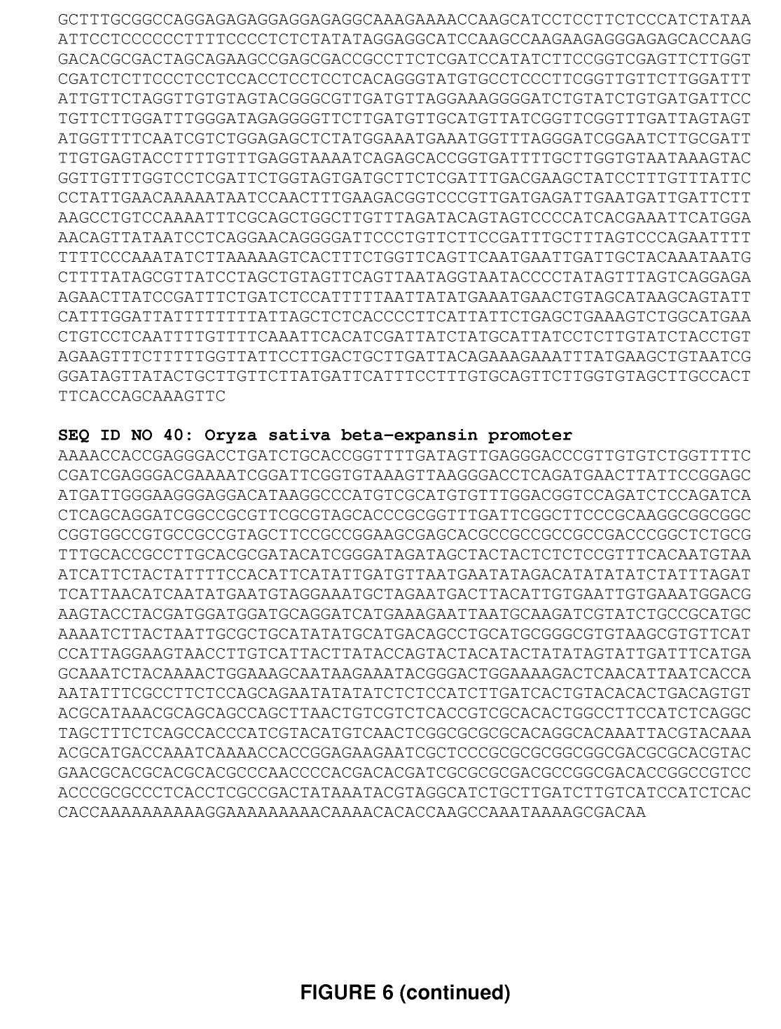

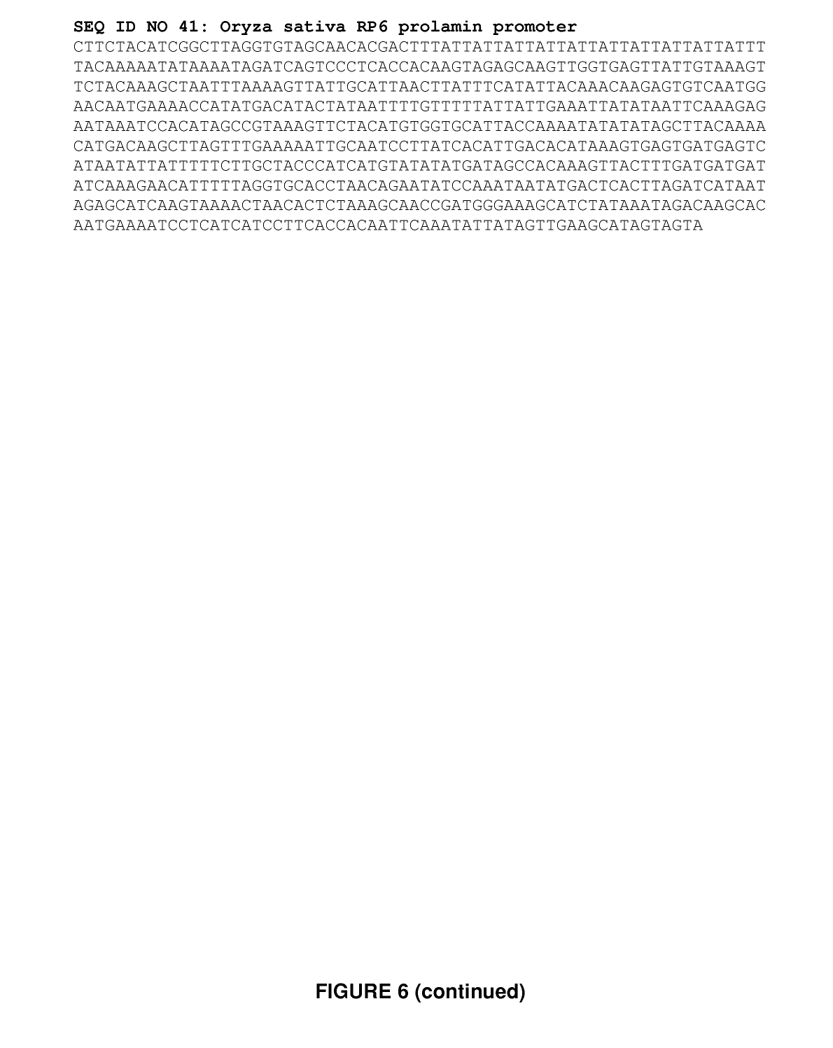

After the LR recombination step, the resulting expression vectors pGOS2::MYB-TF, pProl::MYB-TF, and pExp::MYB-TF (FIG. 5) were independently transformed into Agrobacterium strain LBA4044 according to methods well known in the art.

### Example 7

### Plant Transformation

### Rice Transformation

The two Agrobacterium strains each containing an expression vector, were used independently to transform Oryza sativa plants. Mature dry seeds of the rice japonica cultivar Nipponbare were dehusked. Sterilization was carried out by incubating for one minute in 70% ethanol, followed by 30 minutes in 0.2% HgCl₂, followed by a 6 times 15 minutes wash with sterile distilled water. The sterile seeds were then germinated on a medium containing 2,4-D (callus induction medium). After incubation in the dark for four weeks, embryogenic, scutellum-derived calli were excised and propagated on the same medium. After two weeks, the calli were multiplied or propagated by subculture on the same medium for another 2 weeks. Embryogenic callus pieces were sub-cultured on fresh medium 3 days before co-cultivation (to boost cell division activity).

Agrobacterium strain LBA4404 containing the expression vector was used for co-cultivation. Agrobacterium was inoculated on AB medium with the appropriate antibiotics and cultured for 3 days at 28° C. The bacteria were then collected and suspended in liquid co-cultivation medium to a density (OD₆₀₀) of about 1. The suspension was then transferred to a Petri dish and the calli immersed in the suspension for 15 minutes. The callus tissues were then blotted dry on a filter paper and transferred to solidified, co-cultivation medium and incubated for 3 days in the dark at 25° C. Co-cultivated calli were grown on 2,4-D-containing medium for 4 weeks in the dark at 28° C. in the presence of a selection agent. During this period, rapidly growing resistant callus islands developed. After transfer of this material to a regeneration medium and incubation in the light, the embryogenic potential was released and shoots developed in the next four to five weeks. Shoots were excised from the calli and incubated for 2 to 3 weeks on an auxin-containing medium from which they were transferred to soil. Hardened shoots were grown under high humidity and short days in a greenhouse.

Approximately 35 independent T0 rice transformants were generated per construct. The primary transformants were transferred from a tissue culture chamber to a greenhouse. After a quantitative PCR analysis to verify copy number of the T-DNA insert, only single copy transgenic plants that exhibit tolerance to the selection agent were kept for harvest of T1 seed. Seeds were then harvested three to five months after transplanting. The method yielded single locus transformants at a rate of over 50% (Aldemita and Hodges1996, Chan et al. 1993, Hiei et al. 1994).

### Example 8

### Phenotypic Evaluation Procedure

### 6.1 Evaluation Setup

Approximately 35 independent T0 rice transformants were generated. The primary transformants were transferred from a tissue culture chamber to a greenhouse for growing and harvest of T1 seed. Five to seven independent events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The transgenic plants and the corresponding nullizygotes were grown side-by-side at random positions. Greenhouse conditions were of shorts days (12 hours light), 28° C. in the light and 22° C. in the dark, and a relative humidity of 70%.

In case a T2 generation evaluation was performed, the procedure was the same as for the T1 generation but with more individuals per event. From the stage of sowing until the stage of maturity the plants were passed several times through a digital imaging cabinet. At each time point digital images (2048×1536 pixels, 16 million colours) were taken of each plant from at least 6 different angles. Aboveground biomass was deduced from the images using appropriate software.

To measure root-related parameters, plants were grown in specially designed pots with transparent bottoms to allow visualization of the roots. A digital camera recorded images through the bottom of the pot during plant growth. Root features such as total projected area (which can be correlated to total root volume), average diameter and length of roots above a certain thickness threshold (length of thick roots, or thick root length) were deduced from the image using of appropriate software. Changes of these parameters reflect changes in root biomass, for example, increased root area, increased root length or increased number of thick roots are all indicative of increased root biomass.

### 6.2 Statistical Analysis: F-Test

A two factor ANOVA (analysis of variants) was used as a statistical model for the overall evaluation of plant phenotypic characteristics. An F-test was carried out on all the parameters measured of all the plants of all the events transformed with the gene of the present invention. The F-test was carried out to check for an effect of the gene over all the transformation events and to verify for an overall effect of the gene, also known as a global gene effect. The threshold for significance for a true global gene effect was set at a 5% probability level for the F-test. A significant F-test value points to a gene effect, meaning that it is not only the mere presence or position of the gene that is causing the differences in phenotype.

### 6.3 Parameters Measured

Biomass-Related Parameter Measurement

From the stage of sowing until the stage of maturity the plants were passed several times through a digital imaging cabinet. At each time point digital images (2048×1536 pixels, 16 million colours) were taken of each plant from at least 6 different angles.

The plant aboveground area (or leafy biomass) was determined by counting the total number of pixels on the digital images from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground. The above ground area is the area measured at the time point at which the plant had reached its maximal leafy biomass. The early vigour is the plant (seedling) aboveground area three weeks post-germination.

In addition, another parameter was deduced from the images of the aboveground biomass before flowering: the greenness index, which is the proportion (expressed as %) of green and dark green pixels in the last imaging before flowering.

Increase in root biomass is expressed as an increase in total root biomass (measured as maximum biomass of roots observed during the lifespan of a plant); or as an increase in the root/shoot index (measured as the ratio between root mass and shoot mass in the period of active growth of root and shoot); or as an increase in thick roots; or as an increase in thin roots.

Seed-Related Parameter Measurements

The mature primary panicles were harvested, counted, bagged, barcode-labelled and then dried for three days in an oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. The number of filled seeds was determined by counting the number of filled husks that remained after the separation step. The total seed weight per plant was measured by weighing all filled husks harvested from one plant. Total seed number per plant was measured by counting the number of husks harvested from a plant. Thousand Kernel Weight (TKW) is extrapolated from the number of filled seeds counted and their total weight. The Harvest Index (HI) in the present invention is defined as the ratio between the total seed weight per plant and the above ground area (mm²), multiplied by a factor 10⁶. The total number of flowers per panicle as defined in the present invention is the ratio between the total number of seeds and the number of mature primary panicles. The seed fill rate as defined in the present invention is the proportion (expressed as a %) of the number of filled seeds over the total number of seeds (or florets).

### Example 9

### Results of the Phenotypic Evaluation of the Transgenic Rice Plants Comprising the Nucleic Acid Sequence Encoding a MYB-TF Polypeptide Under the Control of a Constitutive Promoter

As presented in Table D, seedling vigor at emergence, number of thick roots, greenness index (before flowering), seed fill rate, total seed weight per plant, number of filled seeds, and harvest index are increased in the transgenic plants constitutively expressing a nucleic acid sequence encoding a MYB-TF polypeptide, compared to control plants, in the T1 generation.

Table D shows the average percentage increase in seedling vigor at emergence, number of thick roots, greenness index (before flowering), seed fill rate, total seed weight per plant, number of filled seeds, and harvest index of the two best independent transgenic events, compared to control plants, in the T1 generation.

| Parameter | % increase |
|---|---|
| Emergence vigor | 65 |
| Thick roots | 17 |
| Seed fill rate | 21 |
| Total seed weight per plant | 80 |
| Number of filled seeds | 77 |
| Harvest index | 54 |
| Greenness index (before flowering) | 5 |

### Example 10

### Results of the Phenotypic Evaluation of the Transgenic Rice Plants Comprising the Nucleic Acid Sequence Encoding a MYB-TF Polypeptide Under the Control of an Endosperm-Specific Promoter

As presented in Table E, the total seed weight per plant, number of filled seeds and harvest index are increased in the transgenic plants with preferentially increased expression of a nucleic acid sequence encoding a MYB-TF polypeptide in the endosperm, compared to control plants, both in the T1 generation and in the T2 generation.

Table E shows the average percentage increase in yield (total seed weight per plant), in the number of filled seeds, and in the harvest index, of independent transgenic events compared to control plants, in the T1 and T2 generation.

| | % increase | % increase |
|---|---|---|
| | in T1 | in T2 |
| Total seed weight per plant | 20 | 27 |
| Number of filled seeds | 18 | 26 |
| Harvest index | 17 | 25 |

The total number of seeds, the fill rate and the seedling vigor at emergence are also increased in the transgenic plants having preferentially increased expression of a nucleic acid sequence encoding a MYB-TF polypeptide in the endosperm (of a plant seed), compared to control plants.

### Example 11

### Results of the Phenotypic Evaluation of the Transgenic Rice Plants Comprising the Nucleic Acid Sequence Encoding a MYB-TF Polypeptide Under the Control of a Promoter for Expression in Young Expanding Tissues

Transgenic plants expressing of a nucleic acid sequence encoding a MYB-TF polypeptide under the control of beta-expansin promoter, for expression in young expanding tissues, show increased harvest index compared to control plants, in the T1 generation (see Table F).

Table F shows the average percentage increase in harvest index of independent transgenic events compared to control plants, in the T1 generation.

| Parameter | % increase |
|---|---|
| Harvest index | 16 |

### Example 12

### Examples of Transformation of Other Crops

### Corn Transformation

Transformation of maize (Zea mays) is performed with a modification of the method described by Ishida et al. (1996) Nature Biotech 14(6): 745-50. Transformation is genotype-dependent in corn and only specific genotypes are amenable to transformation and regeneration. The inbred line A188 (University of Minnesota) or hybrids with A188 as a parent are good sources of donor material for transformation, but other genotypes can be used successfully as well. Ears are harvested from corn plant approximately 11 days after pollination (DAP) when the length of the immature embryo is about 1 to 1.2 mm. Immature embryos are cocultivated with Agrobacterium tumefaciens containing the expression vector, and transgenic plants are recovered through organogenesis. Excised embryos are grown on callus induction medium, then maize regeneration medium, containing the selection agent (for example imidazolinone but various selection markers can be used). The Petri plates are incubated in the light at 25° C. for 2-3 weeks, or until shoots develop. The green shoots are transferred from each embryo to maize rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Wheat Transformation

Transformation of wheat is performed with the method described by Ishida et al. (1996) Nature Biotech 14(6): 745-50. The cultivar Bobwhite (available from CIMMYT, Mexico) is commonly used in transformation. Immature embryos are co-cultivated with Agrobacterium tumefaciens containing the expression vector, and transgenic plants are recovered through organogenesis. After incubation with Agrobacterium, the embryos are grown in vitro on callus induction medium, then regeneration medium, containing the selection agent (for example imidazolinone but various selection markers can be used). The Petri dishes are incubated in the light at 25° C. for 2-3 weeks, or until shoots develop. The green shoots are transferred from each embryo to rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Soybean Transformation

Soybean is transformed according to a modification of the method described in the Texas A&M patent U.S. Pat. No. 5,164,310. Several commercial soybean varieties are amenable to transformation by this method. The cultivar Jack (available from the Illinois Seed foundation) is commonly used for transformation. Soybean seeds are sterilised for in vitro sowing. The hypocotyl, the radical and one cotyledon are excised from seven-day old young seedlings. The epicotyl and the remaining cotyledon are further grown to develop axillary nodes. These axillary nodes are excised and incubated with Agrobacterium tumefaciens containing the expression vector. After the cocultivation treatment, the explants are washed and transferred to selection media. Regenerated shoots are excised and placed on a shoot elongation medium. Shoots no longer than 1 cm are placed on rooting medium until roots develop. The rooted shoots are transplanted to soil in the greenhouse. T1 seeds are produced from plants that exhibit tolerance to the selection agent and that contain a single copy of the T-DNA insert.

### Rapeseed/Canola Transformation

Cotyledonary petioles and hypocotyls of 5-6 day old young seedling are used as explants for tissue culture and transformed according to Babic et al. (1998, Plant Cell Rep 17: 183-188). The commercial cultivar Westar (Agriculture Canada) is the standard variety used for transformation, but other varieties can also be used. Canola seeds are surface-sterilized for in vitro sowing. The cotyledon petiole explants with the cotyledon attached are excised from the in vitro seedlings, and inoculated with Agrobacterium (containing the expression vector) by dipping the cut end of the petiole explant into the bacterial suspension. The explants are then cultured for 2 days on MSBAP-3 medium containing 3 mg/l BAP, 3% sucrose, 0.7% Phytagar at 23° C., 16 hr light. After two days of co-cultivation with Agrobacterium, the petiole explants are transferred to MSBAP-3 medium containing 3 mg/l BAP, cefotaxime, carbenicillin, or timentin (300 mg/l) for 7 days, and then cultured on MSBAP-3 medium with cefotaxime, carbenicillin, or timentin and selection agent until shoot regeneration. When the shoots are 5-10 mm in length, they are cut and transferred to shoot elongation medium (MSBAP-0.5, containing 0.5 mg/l BAP). Shoots of about 2 cm in length are transferred to the rooting medium (MS0) for root induction. The rooted shoots are transplanted to soil in the greenhouse. T1 seeds are produced from plants that exhibit tolerance to the selection agent and that contain a single copy of the T-DNA insert.

### Alfalfa Transformation

A regenerating clone of alfalfa (Medicago sativa) is transformed using the method of (McKersie et al., 1999 Plant Physiol 119: 839-847). Regeneration and transformation of alfalfa is genotype dependent and therefore a regenerating plant is required. Methods to obtain regenerating plants have been described. For example, these can be selected from the cultivar Rangelander (Agriculture Canada) or any other commercial alfalfa variety as described by Brown DCW and A Atanassov (1985. Plant Cell Tissue Organ Culture 4: 111-112). Alternatively, the RA3 variety (University of Wisconsin) has been selected for use in tissue culture (Walker et al., 1978 μm J Bot 65:654-659). Petiole explants are cocultivated with an overnight culture of Agrobacterium tumefaciens C58C1 pMP90 (McKersie et al., 1999 Plant Physiol 119: 839-847) or LBA4404 containing the expression vector. The explants are cocultivated for 3 d in the dark on SH induction medium containing 288 mg/L Pro, 53 mg/L thioproline, 4.35 g/L K2SO4, and 100 μm acetosyringinone. The explants are washed in half-strength Murashige-Skoog medium (Murashige and Skoog, 1962) and plated on the same SH induction medium without acetosyringinone but with a suitable selection agent and suitable antibiotic to inhibit Agrobacterium growth. After several weeks, somatic embryos are transferred to BOi2Y development medium containing no growth regulators, no antibiotics, and 50 g/L sucrose. Somatic embryos are subsequently germinated on half-strength Murashige-Skoog medium. Rooted seedlings were transplanted into pots and grown in a greenhouse. T1 seeds are produced from plants that exhibit tolerance to the selection agent and that contain a single copy of the T-DNA insert.

1. A method for increasing plant yield relative to control plants, comprising increasing expression in a plant of a nucleic acid sequence encoding a MYB domain transcription factor (MYB-TF) polypeptide, and selecting for plants having increased yield.

2. The method of claim 1, wherein the expression is preferentially increased in the endosperm of a plant seed.

3. The method of claim 1, wherein said MYB-TF polypeptide is any polypeptide comprising from N-terminus to C-terminus (i) an R2R3 MYB domain comprising two MYB repeats; and (ii) a MYB4 domain having at least 55% sequence identity to the MYB4 domain represented by any one or more of SEQ ID NO: 38, SEQ ID NO: 27, or SEQ ID NO: 28.

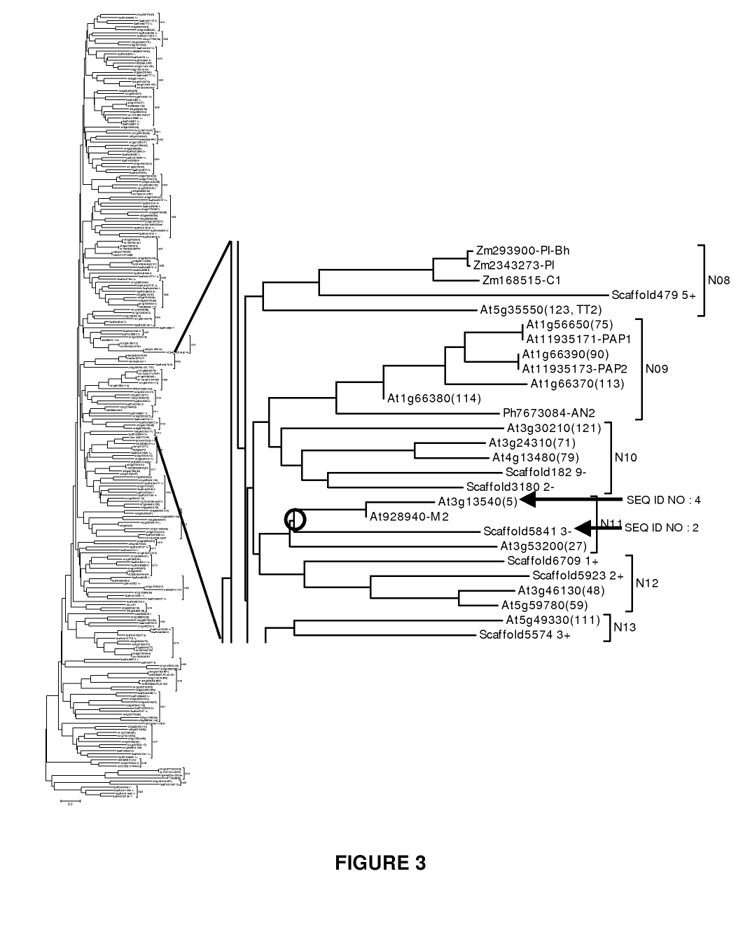

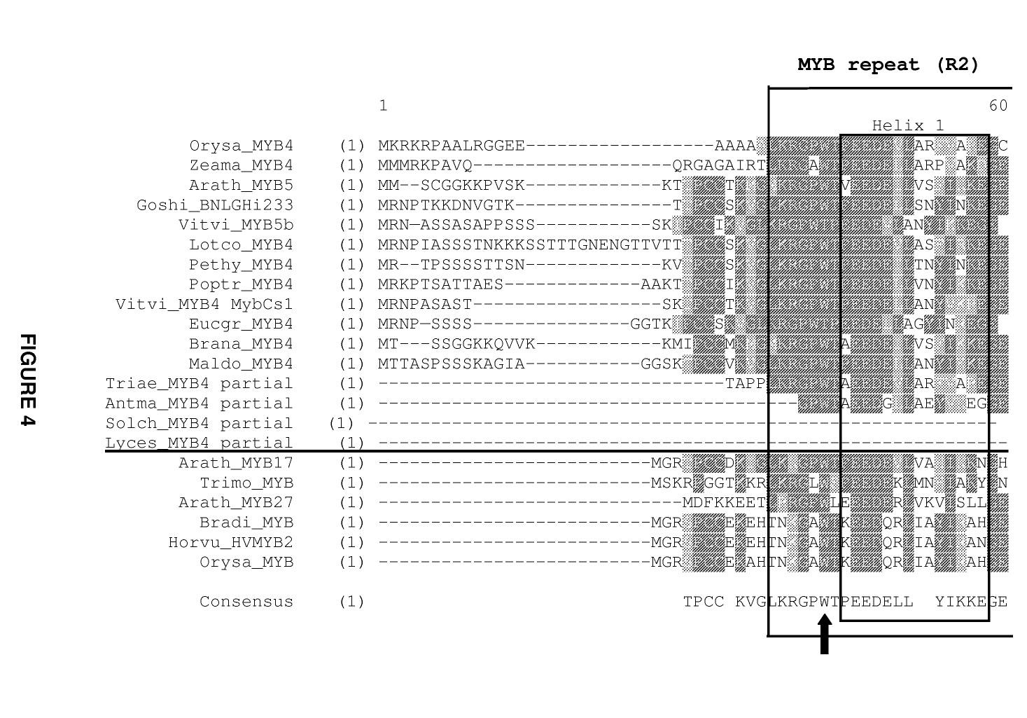

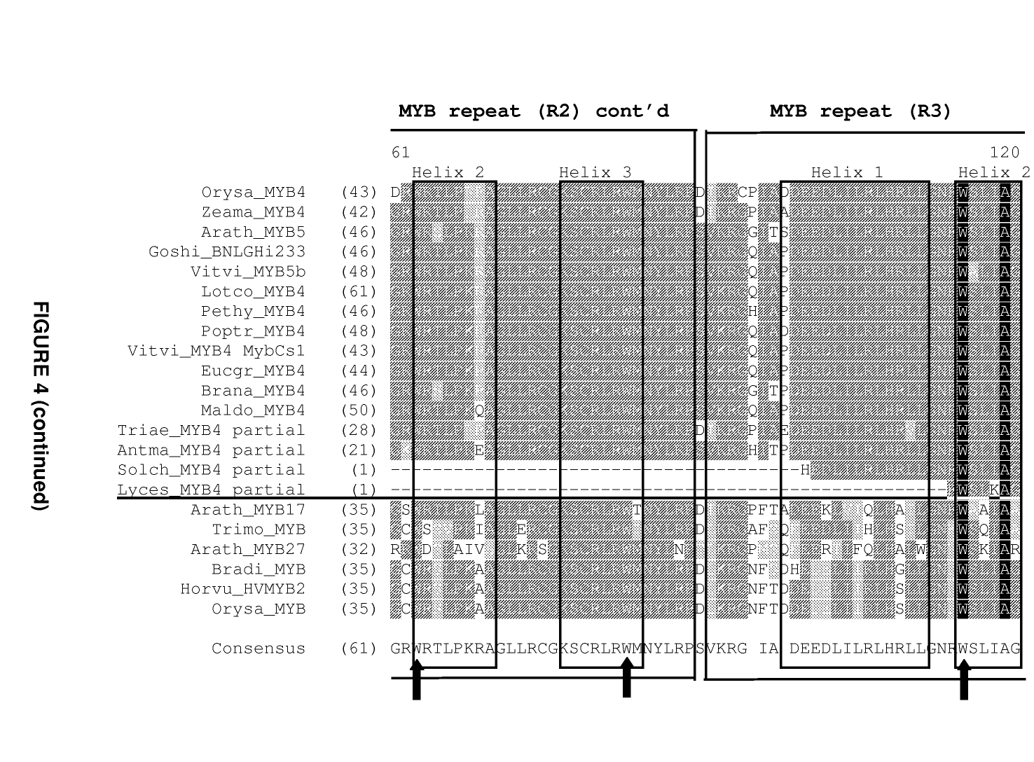

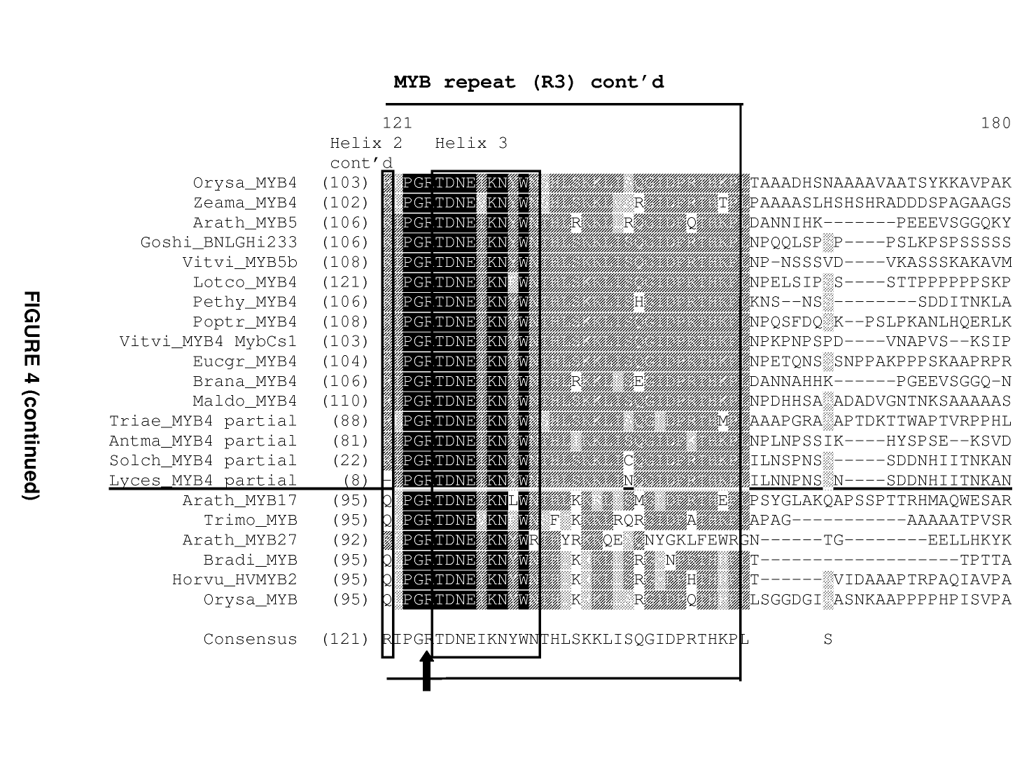

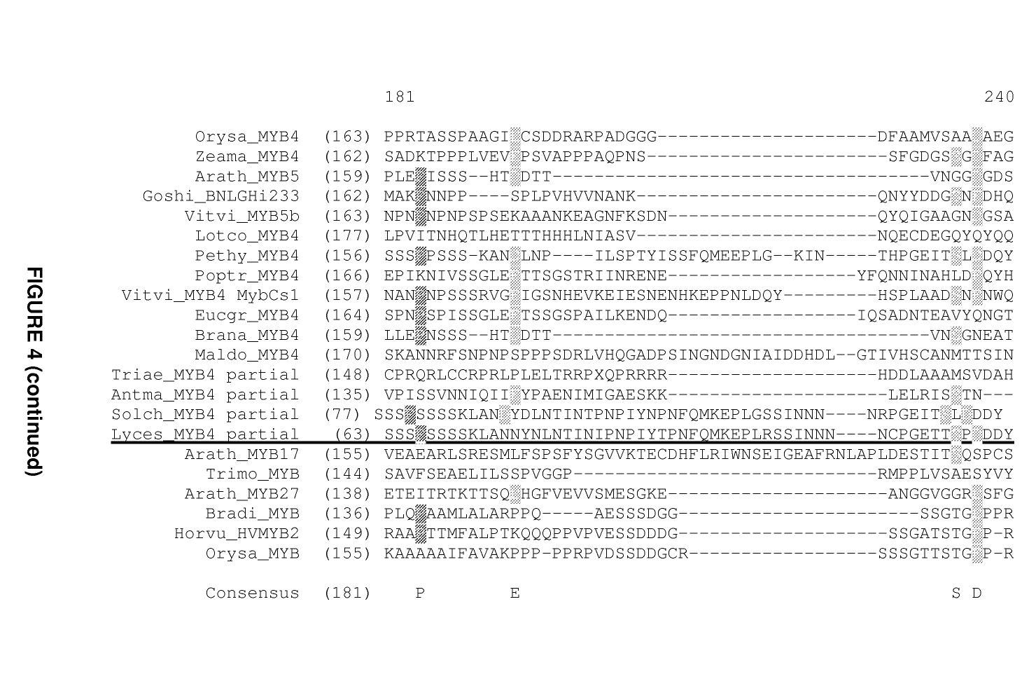

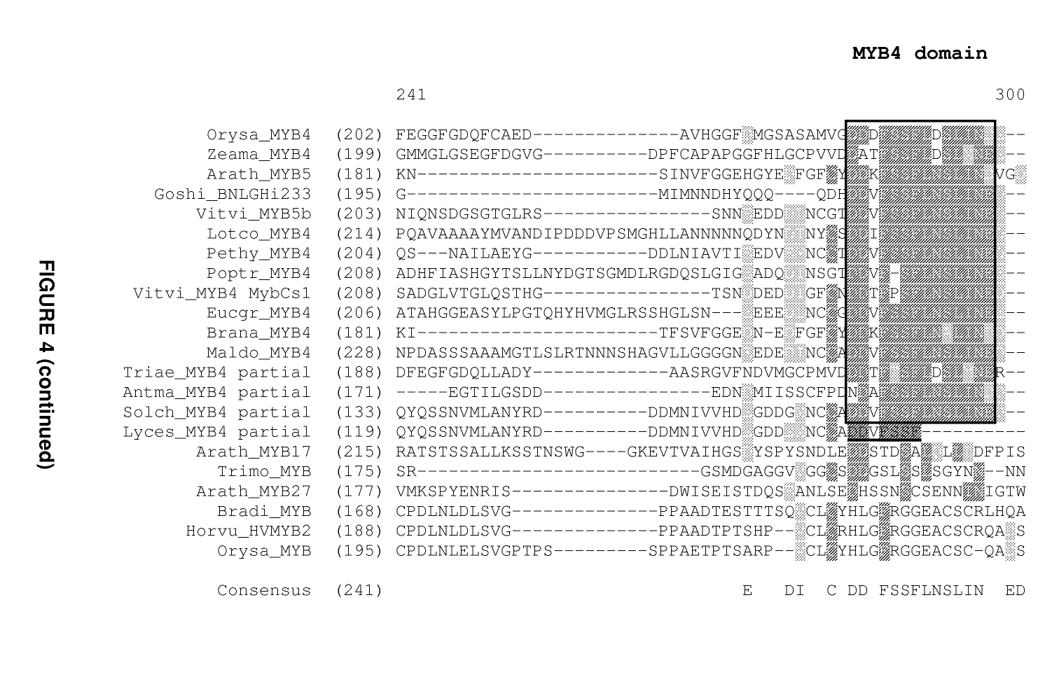

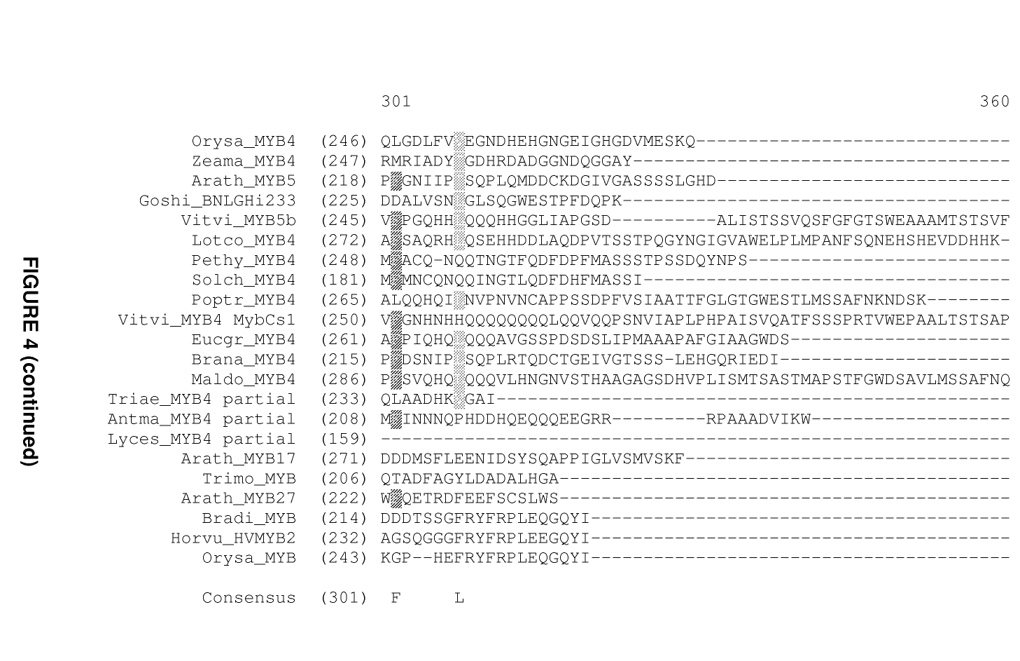

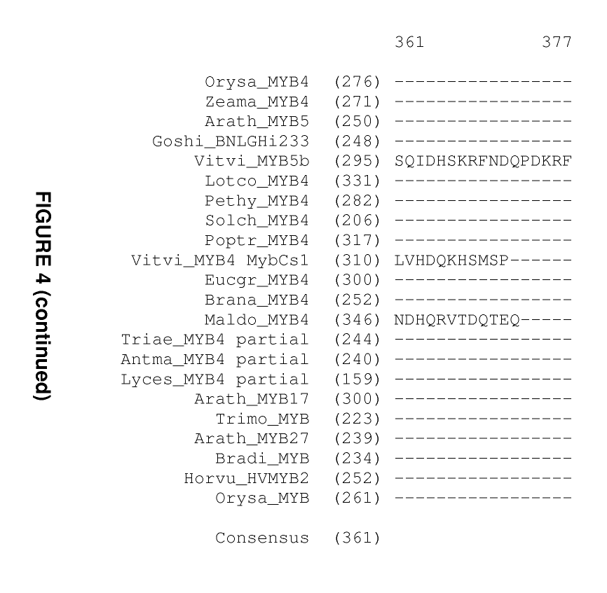

4. The method of claim 1, wherein said MYB-TF polypeptide is any polypeptide sequence which, when used in the construction of a MYB phylogenetic tree such as the one depicted in FIG. 3, tends to cluster with the group of polypeptide sequences comprising the sequences represented by SEQ ID NO: 2 and SEQ ID NO: 4, rather than with any other group, as indicated in FIG. 3.

5. The method of claim 1, wherein said MYB-TF polypeptide is any polypeptide sequence with at least 35% sequence identity to the polypeptide sequence as represented by SEQ ID NO: 2.

6. The method of claim 1, wherein said increased expression is effected by introducing a genetic modification, in the locus of a gene encoding a MYB-TF polypeptide, in a plant.

7. The method of claim 6, wherein said genetic modification is effected by any one or more of: T-DNA activation, TILLING and homologous recombination.

8. A method for increasing plant yield relative to control plants, comprising introducing and expressing in a plant, a nucleic acid sequence encoding a MYB-TF polypeptide.

9. The method of claim 8, wherein said nucleic acid sequence is operably linked to a constitutive promoter or a GOS2 promoter.

10. The method of claim 1, wherein said plant yield is one or more of: (i) increased seedling vigor at emergence; (ii) increased root biomass; (iii) increased greenness index (before flowering); (iv) increased seed fill rate; (v) increased total seed weight per plant; (vi) increased number of filled seeds; or (vii) increased harvest index.

11. A method for increasing plant yield relative to control plants, comprising introducing and increasing expression of a nucleic acid sequence encoding a MYB-TF polypeptide, in the endosperm of a plant seed.

12. The method of claim 11, wherein said nucleic acid sequence is operably linked to an endosperm-specific promoter or a prolamin promoter.

13. The method of claim 11, wherein said plant yield is one or more of: (i) increased total seed weight per plant; (ii) increased number of filled seeds; or (iii) increased harvest index.

14. The method of claim 1, wherein said nucleic acid sequence is a portion or an allelic variant or a splice variant or a sequence capable of hybridising to a nucleic acid sequence as given in Table A, wherein said portion, allelic variant, splice variant or hybridising sequence encodes a MYB-TF polypeptide.

15. The method of claim 14, wherein said portion, allelic variant, splice variant or hybridising sequence encodes an orthologue or paralogue of a MYB-TF polypeptide.

16. The method of claim 1, wherein said nucleic acid sequence encoding a MYB-TF polypeptide is of plant origin, from a monocotyledonous plant from the family Poaceae, from the genus Oryza, or from Oryza sativa.

17. Plants, plant parts, or plant cells obtainable by the method of claim 1, wherein said plants, or parts or cells thereof, comprises an isolated nucleic acid encoding a MYB-TF polypeptide, which isolated nucleic acid is operably linked to a constitutive promoter, or to an endosperm-specific promoter.

18. A construct comprising:
a. a nucleic acid sequence encoding a MYB-TF polypeptide;
b. one or more control sequences capable of driving expression in a plant, plant part or plant cell, of the nucleic acid sequence of (a); and optionally
c. a transcription termination sequence.

19. The construct of claim 18, wherein one of said control sequences is one of: (i) a constitutive promoter or a GOS2 promoter; or (ii) an endosperm-specific promoter or a prolamin promoter.

20. A method for increasing plant yield, comprising transforming the construct of claim 18 into a plant.

21. A plant, plant part or a plant cell transformed with the construct of claim 19.

22. A method for the production of a transgenic plant having increased yield relative to control plants, comprising:
(i) introducing and expressing in a plant, plant part or a plant cell a nucleic acid sequence encoding a MYB-TF polypeptide, wherein the expression of said nucleic acid sequence is driven by a constitutive promoter, or by an endosperm-specific promoter; and
(ii) cultivating the plant part or plant cell under conditions promoting plant growth and development.

23. A transgenic plant having increased yield relative to control plants, said increased yield resulting from increased expression in a plant of a nucleic acid transgene encoding a MYB-TF polypeptide.

24. The transgenic plant of claim 23, wherein said expression is preferentially increased in the endosperm of a plant seed.

25. The transgenic plant of claim 23, wherein said plant is a crop plant or a monocotyledonous plant or a cereal, such as rice, maize, wheat, barley, millet, rye, triticale, sorghum and oats.

26. Harvestable parts of the plant claim 25, wherein said harvestable parts are seeds.

27. Products derived from the plant of claim 25 and/or from harvestable parts of said plant.

28. (canceled)

29. The method of claim 8, wherein said plant yield is one or more of: (i) increased seedling vigor at emergence; (ii) increased number of thick roots; (iii) increased greenness index (before flowering); (iv) increased seed fill rate; (v) increased total seed weight per plant; (vi) increased number of filled seeds; or (vii) increased harvest index.

30. (canceled)

31. The method of claim 20, wherein said increased plant yield is one or more of: (i) increased total seed weight per plant; (ii) increased number of filled seeds; or (iii) increased harvest index.


SEQUENCE LISTING (42 sequences)

SEQ ID NO: 1 - DNA, 828 bp, Oryza sativa
atgaagcggaagcggccggcggcgctgcgcggcggggaggaggcggcggcggcggcgctgaagcgtgggccatggacgcccgaggaggacgaggtgctggcgcggttcgtggcgcgggaggggtgcgaccggtggcgcacgctgccgcggcgcgcgggcctgctgcgctgcggcaagagctgccgcctccggtggatgaactacctccgccccgacatcaagcgctgccccatcgccgacgacgaggaggacctcatcctccgcctccaccgcctcctcggcaaccggtggtcgctgatcgccgggaggttgccggggcgcacggacaacgagatcaagaactactggaactcgcatctcagcaagaagctcatcgcgcagggcatcgacccgcggacgcacaagccgctgacggccgccgccgatcactccaacgccgccgctgccgtcgccgccacttcttacaagaaggcggtgccggccaagccgccgaggacggcatcctcgccggccgctggcattgagtgcagcgacgatcgtgcccgaccggccgacggtggcggtgacttcgcagcgatggtgagcgccgccgatgccgagggattcgaaggaggatttggcgatcagttctgtgccgaggatgcagttcatggtggcttcgacatgggttctgcttccgccatggtgggtgacgacgacttctcctcgtttcttgattctctgatcaacgacgagcagttaggcgatctcttcgtcgtcgagggcaacgatcacgagcatggcaatggtgagattggtcatggagacgtcatggaatccaaacagtaa

SEQ ID NO: 2 - protein, 275 aa, Oryza sativa
MKRKRPAALRGGEEAAAAALKRGPWTPEEDEVLARFVAREGCDRWRTLPRRAGLLRCGKSCRLRWMNYLRPDIKRCPIADDEEDLILRLHRLLGNRWSLIAGRLPGRTDNEIKNYWNSHLSKKLIAQGIDPRTHKPLTAAADHSNAAAAVAATSYKKAVPAKPPRTASSPAAGIECSDDRARPADGGGDFAAMVSAADAEGFEGGFGDQFCAEDAVHGGFDMGSASAMVGDDDFSSFLDSLINDEQLGDLFVVEGNDHEHGNGEIGHGDVMESKQ

SEQ ID NO: 3 - DNA, 750 bp, Arabidopsis thaliana
atgatgtcatgtggtgggaagaagccagtgtctaagaaaacaacgccgtgttgcacgaagatggggatgaagagaggaccatggacggtggaggaagacgagattcttgtgagcttcattaagaaagaaggtgaaggacggtggcgatcgcttcctaagagagctggtttactcagatgtggaaagagctgtcgtctacggtggatgaactatctccgaccctcggttaaacgtggaggaattacgtcggacgaggaagatctcatcctccgtcttcaccgcctcctcggcaacaggtggtcattgatcgcgggaaggataccgggaaggactgataatgaaattaagaactattggaacactcatcttcgtaagaaacttttaaggcaaggaattgatcctcaaacccacaagcctcttgatgcaaacaacatccataaaccagaagaagaagtttccggtggacaaaagtaccctctagagcctatttctagttctcatactgatgataccactgttaatggcggggatggagatagcaagaacagtatcaatgtctttggtggtgaacacggctacgaagactttggtttctgctacgacgacaagttctcatcgtttcttaattcgctcatcaacgatgttggtgatccttttggtaatattatcccaatatctcaacctttgcagatggatgattgtaaggatgggattgttggagcgtcgtcttctagcttaggacatgactag

SEQ ID NO: 4 - protein, 249 aa, Arabidopsis thaliana
MMSCGGKKPVSKKTTPCCTKMGMKRGPWTVEEDEILVSFIKKEGEGRWRSLPKRAGLLRCGKSCRLRWMNYLRPSVKRGGITSDEEDLILRLHRLLGNRWSLIAGRIPGRTDNEIKNYWNTHLRKKLLRQGIDPQTHKPLDANNIHKPEEEVSGGQKYPLEPISSSHTDDTTVNGGDGDSKNSINVFGGEHGYEDFGFCYDDKFSSFLNSLINDVGDPFGNIIPISQPLQMDDCKDGIVGASSSSLGHD

SEQ ID NO: 5 - DNA, 744 bp, Gossypium hirsutum
atgagaaaccctacgaaaaaagacaacgttggaaccaagacgacgccgtgttgcagcaaagtagggttgaaaagagggccatggacgccggaagaagacgagttattatctaattacatcaacaaagaaggcgaaggacggtggcggactttacctaaacgggctggtcttctccgatgcggaaaaagctgtcgtctccgatggatgaattatctccgaccttctgttaaacgtggccagattgctcccgatgaagaagatctcatcctccgccttcaccgccttttagggaatcggtggtcactaatagctggaagaataccagggcgtacagataacgaaataaagaactactggaatactcacctgagtaagaaattgataagtcaagggatcgatcctcgaacgcacaagcctttaaaccctcaacaactctcaccatcaccaccatcactcaaaccttcaccttcttcatcatcatccatggcgaaaccaaacaatcctccttcccctcttcctgttcatgtcgtcaacgctaacaagcaaaattattatgacgatgggagcaatgaagaccatcaagggatgatcatgaacaatgaccattatcagcaacaacaagatcatgatgatgtgttctcttcttttctgaattcattgataaatgaagatgatgatgcattggtttcgaatttgggactctcacaaggatgggaatctactcccttcgatcaaccaaaatga

SEQ ID NO: 6 - protein, 247 aa, Gossypium hirsutum
MRNPTKKDNVGTKTTPCCSKVGLKRGPWTPEEDELLSNYINKEGEGRWRTLPKRAGLLRCGKSCRLRWMNYLRPSVKRGQIAPDEEDLILRLHRLLGNRWSLIAGRIPGRTDNEIKNYWNTHLSKKLISQGIDPRTHKPLNPQQLSPSPPSLKPSPSSSSSMAKPNNPPSPLPVHVVNANKQNYYDDGSNEDHQGMIMNNDHYQQQQDHDDVFSSFLNSLINEDDDALVSNLGLSQGWESTPFDQPK

SEQ ID NO: 7 - DNA, 993 bp, Lotus corniculatus
atgaggaacccgatagcatcatcatcaacgaacaagaagaagagcagtactacaactggtaatgaaaatggcacgactgttactactactccgtgctgcagcaaagttgggttgaagagagggccatggacaccggaggaagacgaggtgttggccagtttcataaggaaagaaggcgaaggtcgctggagaacgcttccaaaaagagccggcctcctccgctgcggtaagagctgccgcctccgctggatgaactacctccgcccctctgtcaaacgcggccagatcgcccccgacgaagaagatctcatcctccgccttcaccgcctcctcggcaaccggtggtctttgatagcgggaagaattccgggaagaactgataatgagataaagaacttctggaacacccatctcagcaagaagcttatcagccaagggattgatccaagaacccacaagcctctcaacccagaactatctattccctcttcttccactactccaccaccaccaccaccttccaagcctcttcctgtgatcacaaatcaccaaacacttcatgaaactactactcatcatcatctcaatattgctagtgtcaaccaagaatgtgatgagggtcaatatcaatatcaacaacctcaagcagtagctgctgctgcatatatggtagctaatgatattcctgatgatgatgttccttccatgggtcatctacttgccaacaacaacaacaaccaggactacaatgatatcaattattgttctgatgatatcttttcttccttcctcaattctctgatcaatgaggatgctttctctgcccagcgccacctgcaatcggagcatcatgatgaccttgcacaggatcctgtaacttcatcaacaccacaaggttataatggcattggtgttgcatgggaattgcccctcatgcctgctaatttcagccaaaatgaacacagccatgaggttgatgatcatcacaaatag

SEQ ID NO: 8 - protein, 330 aa, Lotus corniculatus
MRNPIASSSTNKKKSSTTTGNENGTTVTTTPCCSKVGLKRGPWTPEEDEVLASFIRKEGEGRWRTLPKRAGLLRCGKSCRLRWMNYLRPSVKRGQIAPDEEDLILRLHRLLGNRWSLIAGRIPGRTDNEIKNFWNTHLSKKLISQGIDPRTHKPLNPELSIPSSSTTPPPPPPSKPLPVITNHQTLHETTTHHHLNIASVNQECDEGQYQYQQPQAVAAAAYMVANDIPDDDVPSMGHLLANNNNNQDYNDINYCSDDIFSSFLNSLINEDAFSAQRHLQSEHHDDLAQDPVTSSTPQGYNGIGVAWELPLMPANFSQNEHSHEVDDHHK

SEQ ID NO: 9 - DNA, 915 bp, Medicago truncatula
atgatgaatctatcttcatcaacagatcaagacaaatcagaaacaacaaattcaaattcaaattcaaatccaaacaaaaaacagaagacaacaacaactacaccttgttgcagtaaagtagggttgaagagaggaccgtggactgtagaagaagatgaagttttatcagaatacatcaaaaaagaaggtgaaggtcgttggagaactcttcccaaacgagctggtcttctccgttgcggcaaaagctgtcgtctccgttggatgaattatcttcgtccatccgtcaaacgcggtcagatcgctcctgatgaagaagatctcatcctccgcctccaccgtcttctcggcaacaggtggtcattgatagctgggagaattccaggaagaaccgataatgagattaaaaattattggaacacacatctcagcaagaaacttattaaccaagggattgatcccagaactcacaagcctcttaataacccatcttcttcttcttcaataatcccaaatactaatcaccaaacaagtactcctttttttgcaccatcttcttctgatcatcatcatccaattactatcactaacaatgaacctgatccctctcaaacacaacaagctcagggttgtggtaacttggttaatgatgtttctgcaatggataatcatcatgtgcttaccaacaacaacagaggggattatggagacgacaatggtaataacaattacggcggtgatgatgtgtttacttcatttctggattcattgatcaacgacgatgcatttgctgcacacagatcagagaatgatcctttgatcttatctgctgcaccaccagctctgtgggaatcgccgccactcatgatgacatctactcataatttcacccaaaaccaagacgggaaagcttcttag

SEQ ID NO: 10 - protein, 304 aa, Medicago truncatula
MMNLSSSTDQDKSETTNSNSNSNPNKKQKTTTTTPCCSKVGLKRGPWTVEEDEVLSEYIKKEGEGRWRTLPKRAGLLRCGKSCRLRWMNYLRPSVKRGQIAPDEEDLILRLHRLLGNRWSLIAGRIPGRTDNEIKNYWNTHLSKKLINQGIDPRTHKPLNNPSSSSSIIPNTNHQTSTPFFAPSSSDHHHPITITNNEPDPSQTQQAQGCGNLVNDVSAMDNHHVLTNNNRGDYGDDNGNNNYGGDDVFTSFLDSLINDDAFAAHRSENDPLILSAAPPALWESPPLMMTSTHNFTQNQDGKAS

SEQ ID NO: 11 - DNA, 846 bp, Petunia hybrida
atgagaaccccatcatcatcatcaacaacaagcaacaaagtaacaccatgttgtagcaaggtagggttaaaaagaggtccatggacaccagaagaagatgaaatattgacaaattatataaataaagaaggagaaggacggtggcgaacgttgccaaagaaggcggggctcctccgttgtggaaaaagctgccgccttcggtggatgaattacctccgtccttcagttaaacgtggccatattgcacctgatgaagaagatctcattcttcgcctccatcgtctccttggcaacaggtggtccttgatagctgggagaattccagggagaacagataatgagataaagaattattggaacactcaccttagcaagaagttaatcagtcacggaatagatcctagaactcacaagccattgaaaaactctaattcctcaagtgatgatattaccaacaaattagcttcttcttctcctccttcatcttcaaaagcaaatgatctcaatccaattctaagccccacttacatttctagttttcaaatggaagaaccattaggaaaaatcaatactcacccgggagaaattactagtcttgatgatcaatatcaaagtaacgcgattcttgccgagtatggtgatgatctgaatattgcggttactattgaggaagatgttgaaatgaattgttgcacggatgatgttttctcctctttcttgaattctttgatcaatgaagatatgttcgcttgccaaaaccaacaaaccaacgggacgttccaagattttgatcctttcatggcttcatcatctacaccttcatctgatcaatataatcccagttag

SEQ ID NO: 12 - protein, 281 aa, Petunia hybrida
MRTPSSSSTTSNKVTPCCSKVGLKRGPWTPEEDEILTNYINKEGEGRWRTLPKKAGLLRCGKSCRLRWMNYLRPSVKRGHIAPDEEDLILRLHRLLGNRWSLIAGRIPGRTDNEIKNYWNTHLSKKLISHGIDPRTHKPLKNSNSSSDDITNKLASSSPPSSSKANDLNPILSPTYISSFQMEEPLGKINTHPGEITSLDDQYQSNAILAEYGDDLNIAVTIEEDVEMNCCTDDVFSSFLNSLINEDMFACQNQQTNGTFQDFDPFMASSSTPSSDQYNPS

SEQ ID NO: 13 - DNA, 951 bp, Populus tremuloides
atgagaaagccaacttcagctacaacagcagaatcagcagcaaagaccaccccatgctgcatcaaggtagggttaaagagaggaccgtggacaccagaagaagatgaactccttgttaactatatcaagaaagaaggtgaaggccggtggcgaactttacccaagaaagccggtctcctccgctgtggcaagagctgccgtctccgctggatgaactacctccgcccttctgttaaacgtggccagatcgccgatgatgaagaagatctcattctccgcctccatcgactcctaggcaataggtggtcattgatagctgggagaattccagggcgtacagataatgaaataaagaattactggaacacgcacctgagtaagaagctgataagccaaggaattgatccaagaacccataagcctctcaatcctcaatcttttgatcaatcaaaaccttctttgccgaaggcgaatctgcaccaagaacgcctaaaagaacctattaagaatattgtttcttctggtttggaagaaactactagtggcagtactcgcataatcaacagagagaatgagtattttcaaaacaacattaatgcccacctggatgaacagtatcacgcagaccattttattgcaagtcatggctatacaagtttgctgaattatgatggtacttctgggatggatttgagaggcgatcaaagtcttggtattggagaagctgatcaagatatcaattccggcaccgacgatgtgttttcgttcctcaattcgctcataaatgaggaggctttgcaacaacatcaaatccttaatgtgcctaatgtgaattgtgcaccacccagttcagatccttttgtttcaattgctgccacaacatttggactcggaactggctgggaatctactctcatgtcttctgcttttaacaaaaatgattccaaatag

SEQ ID NO: 14 - protein, 316 aa, Populus tremuloides
MRKPTSATTAESAAKTTPCCIKVGLKRGPWTPEEDELLVNYIKKEGEGRWRTLPKKAGLLRCGKSCRLRWMNYLRPSVKRGQIADDEEDLILRLHRLLGNRWSLIAGRIPGRTDNEIKNYWNTHLSKKLISQGIDPRTHKPLNPQSFDQSKPSLPKANLHQERLKEPIKNIVSSGLEETTSGSTRIINRENEYFQNNINAHLDEQYHADHFIASHGYTSLLNYDGTSGMDLRGDQSLGIGEADQDINSGTDDVFSFLNSLINEEALQQHQILNVPNVNCAPPSSDPFVSIAATTFGLGTGWESTLMSSAFNKNDSK

SEQ ID NO: 15 - DNA, 936 bp, Vitis vinifera
atgaggaatgcatcctcagcatcagctccaccttcatcttcttcaaaaacaccatgctgcatcaaggttggattgaagagggggccatggacgccggaggaagacgaggttctggccaattacatcaagaaagaaggagaaggccggtggcgcaccctcccgaagcgcgccggtctcctccgctgcggcaagagctgtcgcctccgctggatgaactacctccgcccttccgtcaagcgcggccagatcgcccccgatgaagaggatctcattctccgcctccaccgacttctcggcaacaggtgggctttgattgccggaagaattccgggccggacggataatgagataaaaaactactggaacacacacctgagcaagaagctgatcagccagggaatagatccgagaacccataagccattgaaccctaattcatcatctgttgatgtgaaagcttcttcttcaaaggcaaaagctgttatgaaccctaaccctaaccctaatccttctccttcagaaaaagcagcagccaacaaggaagctgggaacttcaagagtgacaatcagtatcagattggggcagctggcaatgatggcagtgccaatatccagaattcggatggttccgggaccggattgaggagcagcaacaacgaagaagacgatgaccttaactgtggcaccgatgatgtcttctcttcatttttgaactcattgatcaatgaggatgtgtttcctggacagcaccatctccaacaacagcaccatggtggtctcattgcaccgggctccgatgctttgatctctacttcttcagtccagtcgttcgggttcggtaccagctgggaggctgcagccatgacttccacgtctgtttttagccaaatcgatcactccaagaggtttaacgatcaacctgataagcggttctga

SEQ ID NO: 16 - protein, 311 aa, Vitis vinifera
MRNASSASAPPSSSSKTPCCIKVGLKRGPWTPEEDEVLANYIKKEGEGRWRTLPKRAGLLRCGKSCRLRWMNYLRPSVKRGQIAPDEEDLILRLHRLLGNRWALIAGRIPGRTDNEIKNYWNTHLSKKLISQGIDPRTHKPLNPNSSSVDVKASSSKAKAVMNPNPNPNPSPSEKAAANKEAGNFKSDNQYQIGAAGNDGSANIQNSDGSGTGLRSSNNEEDDDLNCGTDDVFSSFLNSLINEDVFPGQHHLQQQHHGGLIAPGSDALISTSSVQSFGFGTSWEAAAMTSTSVFSQIDHSKRFNDQPDKRF

SEQ ID NO: 17 - DNA, 813 bp, Zea mays
atgatgatgcggaagccggcggtgcagcagcgtggcgcgggggcgataaggacgctcaagcggggtgcgtggacgccggaggaggacgagctgctggcgcggcccgtggcgaaggacggggaggggcggtggcgcacgctgccgcggcgggcggggctgcttcgctgcggcaagagctgcaggctccgctggatgaactacctcaggccggacatcaagcgcgggcctatcgccgccgacgaggaggacctcatcctccgcctccaccgcctgctcggaaacaggtggtcgctgatcgctgggcgtctgcctggccgcacggacaacgaggtcaagaactactggaactcgcacctcagcaagaagctcgtcgcgcggggcatcgacccgcgcacgcacacgcctctgcccgcagcagctgcgagcctgcactcgcactcccaccgcgccgacgacgactcaccagcaggagccgccggttcgtctgccgacaagacgccgccgccactggtggaagtcgagccatcggttgcgccgccgccagcgcagccgaactcatctttcggcgacggaagcagcggcgacttcgcggggggcatgatgggtctaggctccgagggcttcgatggagtcggtgatccgttctgtgcaccagctcctggcggcttccacttgggctgccccgtcgtggacgacgccaccttctcctcgttcctcgactcgctgatgaacgaggagcgcatgcgcatcgccgattacattggagatcacagggatgctgatggtgggaacgaccagggtggagcttattag

SEQ ID NO: 18 - protein, 270 aa, Zea mays
MMMRKPAVQQRGAGAIRTLKRGAWTPEEDELLARPVAKDGEGRWRTLPRRAGLLRCGKSCRLRWMNYLRPDIKRGPIAADEEDLILRLHRLLGNRWSLIAGRLPGRTDNEVKNYWNSHLSKKLVARGIDPRTHTPLPAAAASLHSHSHRADDDSPAGAAGSSADKTPPPLVEVEPSVAPPPAQPNSSFGDGSSGDFAGGMMGLGSEGFDGVGDPFCAPAPGGFHLGCPVVDDATFSSFLDSLMNEERMRIADYIGDHRDADGGNDQGGAY

SEQ ID NO: 19 - DNA, 720 bp, Antirrhinum majus
gggccatggacggcggaggaggacgggcttttagcggagtacgtaaaggagggaggtgaggggaagtggcgcacgctgccgaaggaggcggggctgctacggtgcggcaagagctgccgcctccggtggatgaactacctccgtccctccgtcaagcgcggccacatcactcctgacgaggaggatctcatcctccgcctccaccgcctcctcggaaacaggtggtctttaatagcgggaagaatacctgggcggacagataacgagataaagaactactggaacactcacctcaccaaaaaactcataagccaaggaatcgatcccaaaacacacaagccattgaatccacttaaccccagcagcatcaaacattactctccctcagaaaaatcagttgatgttccaatttcctcagtaaacaatatccaaattatagaatatccagctgaaaacatcatgatcggtgcagagtccaagaaattagagttgagaataagtagtactaatgaggggaccatattgggtagtgatgatgaagataatgacatgatcatcagcagttgtttccctgataatgatgccttttcatccttcttgaattcactcattaacgatgatatgttcattaataacaaccaaccccacgatgatcatcaggaacaacaacaagaggaggggagacgccgaccggctgctgcagatgtaataaagtggtga

SEQ ID NO: 20 - protein, 239 aa, Antirrhinum majus
GPWTAEEDGLLAEYVKEGGEGKWRTLPKEAGLLRCGKSCRLRWMNYLRPSVKRGHITPDEEDLILRLHRLLGNRWSLIAGRIPGRTDNEIKNYWNTHLTKKLISQGIDPKTHKPLNPLNPSSIKHYSPSEKSVDVPISSVNNIQIIEYPAENIMIGAESKKLELRISSTNEGTILGSDDEDNDMIISSCFPDNDAFSSFLNSLINDDMFINNNQPHDDHQEQQQEEGRRRPAAADVIKW

SEQ ID NO: 21 - DNA, 476 bp, Lycopersicon esculentum
  misc_feature (11)..(11): n is a, c, g, or t
tggtcattganagcagggngaattcccggaagaacggataacgagataaagaattattggaatacccaccttagcaagaaattaatcaaccaaggaatagatccaagaacacacaaaccattaatattaaacaaccctaattcctctaatagtgatgataatcacattattacaaacaaagctaattcttcttctccttcttcatcctcaaaactagcaaataattataatctcaacacaattaatattccaaaccccatttacactcctaatttccaaatgaaagaacccttaagaagtagcatcaacaataataactgtcctggagaaactacgagtcctgatgatgattatcagtatcagagcagtaatgtgatgcttgccaattatcgcgatgatgatatgaatattgtggttcatgatgagggagatgatgaaatgaattgttgcgcggacgatgtcttctcctccttctt

SEQ ID NO: 22 - protein, 159 aa, Lycopersicon esculentum
  misc_feature (4)..(4): Xaa can be any naturally occurring amino acid
WSLXAGXIPGRTDNEIKNYWNTHLSKKLINQGIDPRTHKPLILNNPNSSNSDDNHIITNKANSSSPSSSSKLANNYNLNTINIPNPIYTPNFQMKEPLRSSINNNNCPGETTSPDDDYQYQSSNVMLANYRDDDMNIVVHDEGDDEMNCCADDVFSSFL

SEQ ID NO: 23 - DNA, 618 bp, Solanum chacoense
cacgaggatctcatcctccgcctccatcgcctcctagggaacaggtggtcattgatagcaggcagaattccaggaagaacagacaacgagataaagaattattggaatactcaccttagcaagaaattaatctgccaaggaatagatccaagaacacacaaaccattaatattaaacagccctaattcctctagtgatgataatcatattattacaaacaaagctaattcttcttctccttcttcatcctcaaaactagcaaatgattatgatctcaacacaattaatactccaaaccccatttacaatcctaatttccaaatgaaagaacccttaggaagtagcatcaacaataataaccgtcctggagaaattacgagtcttgatgatgattatcagtatcagagcagtaatgtgatgcttgccaattatcgcgatgatgatatgaacattgtggttcatgatgagggagacgatggaatgaattgttgcgcggacgatgtcttctcctccttcttgaattctttgatcaatgaagacatgttcatgaattgccaaaatcaacaaatcaatggtacattgcaagattttgaccattttatggcttcatctatatga

SEQ ID NO: 24 - protein, 205 aa, Solanum chacoense
HEDLILRLHRLLGNRWSLIAGRIPGRTDNEIKNYWNTHLSKKLICQGIDPRTHKPLILNSPNSSSDDNHIITNKANSSSPSSSSKLANDYDLNTINTPNPIYNPNFQMKEPLGSSINNNNRPGEITSLDDDYQYQSSNVMLANYRDDDMNIVVHDEGDDGMNCCADDVFSSFLNSLINEDMFMNCQNQQINGTLQDFDHFMASSI

SEQ ID NO: 25 - DNA, 732 bp, Triticum aestivum
  misc_feature (502)..(502): n is a, c, g, or t
acggcgccgccgctgaagcgggggccgtggacggcggaggaggacgaggtgctggcgcggttcgtggcgcgggagggggaggggcggtggcgcacgctgccacggcgggcgggcctgctgcgctgcggcaagagctgccgcctgcggtggatgaactacctccggccggacatcaagcgcggccccatcgccgaggacgaggaggacctcatcctccgcctccaccgcgtcctcggcaaccggtggtcgctgatcgccgggaggctgccggggcgcacggacaacgagatcaagaactactggaactcgcacctcagcaagaagctcatcgcccagggcctcgacccgcggacgcacatgccgctcgccgctgctcccggcagggcggcagctcctaccgacaagacgacgtgggcgccgacggtcagaccgccgcacctctgcccccgccagcgcttgtgctgccgacctcgtctgccgctggaactgacgcgacggccgngacagcccaggcgacggaggcatgacgacctcgccgcggcgatgagcgtcgacgcccatgacttcgaaggtttcggtgatcagcttctcgccgactacgctgcttcccgcggagtcttcaacgacgtgatggggtgtccaatggtggacgacgacaccttcacctcgttcctcgattccttgatgaatgagcgccagttggctgctgatcacaaggttggcgctatctag

SEQ ID NO: 26 - protein, 243 aa, Triticum aestivum
  misc_feature (168)..(168): Xaa can be any naturally occurring amino acid
TAPPLKRGPWTAEEDEVLARFVAREGEGRWRTLPRRAGLLRCGKSCRLRWMNYLRPDIKRGPIAEDEEDLILRLHRVLGNRWSLIAGRLPGRTDNEIKNYWNSHLSKKLIAQGLDPRTHMPLAAAPGRAAAPTDKTTWAPTVRPPHLCPRQRLCCRPRLPLELTRRPXQPRRRRHDDLAAAMSVDAHDFEGFGDQLLADYAASRGVFNDVMGCPMVDDDTFTSFLDSLMNERQLAADHKVGAI

SEQ ID NO: 27 - protein, 14 aa, Artificial sequence
  MYB4 domain
DDXFSSFLNSLINE

SEQ ID NO: 28 - protein, 14 aa, Artificial sequence
  MYB4 domain of Oryza sativa as in SEQ ID NO 2
DDDFSSFLDSLIND

SEQ ID NO: 29 - DNA, 654 bp, Oryza sativa
cttctacatcggcttaggtgtagcaacacgactttattattattattattattattattattattttacaaaaatataaaatagatcagtccctcaccacaagtagagcaagttggtgagttattgtaaagttctacaaagctaatttaaaagttattgcattaacttatttcatattacaaacaagagtgtcaatggaacaatgaaaaccatatgacatactataattttgtttttattattgaaattatataattcaaagagaataaatccacatagccgtaaagttctacatgtggtgcattaccaaaatatatatagcttacaaaacatgacaagcttagtttgaaaaattgcaatccttatcacattgacacataaagtgagtgatgagtcataatattattttctttgctacccatcatgtatatatgatagccacaaagttactttgatgatgatatcaaagaacatttttaggtgcacctaacagaatatccaaataatatgactcacttagatcataatagagcatcaagtaaaactaacactctaaagcaaccgatgggaaagcatctataaatagacaagcacaatgaaaatcctcatcatccttcaccacaattcaaatattatagttgaagcatagtagta

SEQ ID NO: 30 - protein, 50 aa, Artificial sequence
  primer prm5233
GGGGACAAGTTTGTACAAAAAAGCAGGCTTAAACAATGAAGCGGAAGCGG

SEQ ID NO: 31 - protein, 50 aa, Artificial sequence
  primer prm5234
GGGGACCACTTTGTACAAGAAAGCTGGGTAATCTCCCGAAAGATTACTGT

SEQ ID NO: 32 - DNA, 756 bp, Brassica napus
atgacgtcatctggaggaaagaagcaggtcgtgaagaagatgatcccgtgctgcatgaagatggggatgaagagaggaccatggacggcggaggaggatgagattcttgttagcttcatcaagaaagaaggcgaaggaaggtggagatcgcttcccaagagagctggtttactccgatgtggaaagagctgccgcctacggtggatgaactatctccggccgtctgtaaaacgcggcggtatcactcccgacgaggaagatctcatcctccgccttcatcgcctccttggcaaccggtggtcactgatcgcgggaaggataccgggaaggactgataatgagattaagaactattggaacactcatcttcgtaagaaactcttaagtgaaggcattgatcctcgaacccacaagcctcttgatgcaaacaatgctcatcataaacccggagaagaagtttccggtggacagaatcttctagagcctaattctagttctcacactgatgacaccaccgttaatagcggaaatgaagctaccaagatcactttcagtgtctttggtggtgaagacaacgaagactttggtttctgttacgatgataagttttcttcgtttctaaatgcgcttattaatgatgatccttttgatagtaatattccattgtcccagccgttacggacgcaagattgtactggtgagattgttggaacgtcgtctagcttagaacatggccagagaatagaagatatatga

SEQ ID NO: 33 - protein, 251 aa, Brassica napus
MTSSGGKKQVVKKMIPCCMKMGMKRGPWTAEEDEILVSFIKKEGEGRWRSLPKRAGLLRCGKSCRLRWMNYLRPSVKRGGITPDEEDLILRLHRLLGNRWSLIAGRIPGRTDNEIKNYWNTHLRKKLLSEGIDPRTHKPLDANNAHHKPGEEVSGGQNLLEPNSSSHTDDTTVNSGNEATKITFSVFGGEDNEDFGFCYDDKFSSFLNALINDDPFDSNIPLSQPLRTQDCTGEIVGTSSSLEHGQRIEDI

SEQ ID NO: 34 - DNA, 900 bp, Eucalyptus grandis
atgaggaatccgtcgtcgtcgtcgggagggacgaagaccccgtgctgcagcaaggtggggctgaagagggggccctggacgccggaggaggacgagctcctcgccggctacatcaaccgcgagggcgagggccggtggcggaccctccccaagcgggccggcctcctccgctgcggcaagagctgccgcctccgctggatgaactacctccgcccctccgtcaagcgcggccagatcgcccccgacgaggaggacctcatcctccgcctccaccgcctcctcggcaaccggtggtctttgatagctgggagaatcccaggacggactgacaatgagatcaagaactactggaacactcaccttagcaagaagctcatcagtcaaggaattgatccgaggacgcacaagccgctcaatccagaaacccagaactccagcagcaatcctccagcgaaacctcctccttccaaagcggcacctcgccctcgaagccctaaccctagccccatctcttctggcctcgaggaaaccagcagtggcagcccagccatcctcaaagaaaatgatcagatccaaagtgccgataatacggaggctgtgtatcaaaatgggactgcgacagctcacggtggtgaagccagttatttgccgggtacccagcattaccatgtgatgggcttgagaagcagccacggactgtctaatgaggaggaggaggacctcaactgctgcggggacgacgtcttctcatcgttcctgaactccctgatcaacgaggaagcattcccaatccagcaccagctacaacaacaggccgtcggctcctcgccggattcggattctctgattcccatggctgcagcacctgcattcgggatagccgctggctgggactcttag

SEQ ID NO: 35 - protein, 299 aa, Eucalyptus grandis
MRNPSSSSGGTKTPCCSKVGLKRGPWTPEEDELLAGYINREGEGRWRTLPKRAGLLRCGKSCRLRWMNYLRPSVKRGQIAPDEEDLILRLHRLLGNRWSLIAGRIPGRTDNEIKNYWNTHLSKKLISQGIDPRTHKPLNPETQNSSSNPPAKPPPSKAAPRPRSPNPSPISSGLEETSSGSPAILKENDQIQSADNTEAVYQNGTATAHGGEASYLPGTQHYHVMGLRSSHGLSNEEEEDLNCCGDDVFSSFLNSLINEEAFPIQHQLQQQAVGSSPDSDSLIPMAAAPAFGIAAGWDS

SEQ ID NO: 36 - DNA, 1125 bp, Malus domestica
atgaggaacccgtcgtcttcgtcgaaagcaacagcagcagcaagtgctacgatgacaacagcatcgccgtcgtcgagcaaggcggggattgccggagggagtaagacgccgtgttgcgtaaaggtgggtttgaagagggggccgtggactcccgaagaggacgagctgctagccaattacatcaagaaagaaggtgaaggacggtggcggaccctccccaagcaggctgggttgctccgctgcggaaaaagctgtcgcctccgctggatgaactacctccgcccttccgttaagcgcggccagatcgcccccgatgaagaagatctcattctccgcctccatcgcctcctcggcaatcggtggtctttgatagctgggaggattccaggtcgtacggacaatgagataaagaactactggaacacacacctgagcaagaagctgataagccaaggcatagatcccagaacccacaagcctctcaatccagatcatcactctgctgctgccgatgctgatgtgggcaacacaaataaatcagctgctgctgctgcttcttccaaagccaataaccgattctcaaaccctaatcctagtcctcctccttctgatcgtcttgtccatcaaggagcggatcccagtatcaacggtaatgatggaaacatcgcaattgatgatcatgatctgggcactatagtccatagctgtgcaaacatgaccacgtccattaacaatcccgatgcttcttcttcggccgcagcaatgggcactttgagtttaaggaccaacaacaatagccacgctggagtactacttgggggaggaggaaatgaagaggacgaggacatcaactgttgtgcggacgatgtcttctcttcgtttctgaattcgttgatcaacgaggatccattttctgtacaacaccaattgcaacaacaggtactgcacaatgggaatgttagtacacacgcagctggtgctggttccgaccacgttcctttgatttctatgactagtgctagtactatggcgccgtcaacatttggctgggactctgctgtgctcatgtcttctgctttcaaccaaaatgatcaccagagggttactgatcaaacggagcagtag

SEQ ID NO: 37 - protein, 374 aa, Malus domestica
MRNPSSSSKATAAASATMTTASPSSSKAGIAGGSKTPCCVKVGLKRGPWTPEEDELLANYIKKEGEGRWRTLPKQAGLLRCGKSCRLRWMNYLRPSVKRGQIAPDEEDLILRLHRLLGNRWSLIAGRIPGRTDNEIKNYWNTHLSKKLISQGIDPRTHKPLNPDHHSAAADADVGNTNKSAAAAASSKANNRFSNPNPSPPPSDRLVHQGADPSINGNDGNIAIDDHDLGTIVHSCANMTTSINNPDASSSAAAMGTLSLRTNNNSHAGVLLGGGGNEEDEDINCCADDVFSSFLNSLINEDPFSVQHQLQQQVLHNGNVSTHAAGAGSDHVPLISMTSASTMAPSTFGWDSAVLMSSAFNQNDHQRVTDQTEQ

SEQ ID NO: 38 - protein, 14 aa, Artificial sequence
  MYB4 domain
DDXFSSFLNSLINE

SEQ ID NO: 39 - DNA, 2194 bp, Oryza sativa
aatccgaaaagtttctgcaccgttttcaccccctaactaacaatatagggaacgtgtgctaaatataaaatgagaccttatatatgtagcgctgataactagaactatgcaagaaaaactcatccacctactttagtggcaatcgggctaaataaaaaagagtcgctacactagtttcgttttccttagtaattaagtgggaaaatgaaatcattattgcttagaatatacgttcacatctctgtcatgaagttaaattattcgaggtagccataattgtcatcaaactcttcttgaataaaaaaatctttctagctgaactcaatgggtaaagagagagattttttttaaaaaaatagaatgaagatattctgaacgtattggcaaagatttaaacatataattatataattttatagtttgtgcattcgtcatatcgcacatcattaaggacatgtcttactccatcccaatttttatttagtaattaaagacaattgacttatttttattatttatcttttttcgattagatgcaaggtacttacgcacacactttgtgctcatgtgcatgtgtgagtgcacctcctcaatacacgttcaactagcaacacatctctaatatcactcgcctatttaatacatttaggtagcaatatctgaattcaagcactccaccatcaccagaccacttttaataatatctaaaatacaaaaaataattttacagaatagcatgaaaagtatgaaacgaactatttaggtttttcacatacaaaaaaaaaaagaattttgctcgtgcgcgagcgccaatctcccatattgggcacacaggcaacaacagagtggctgcccacagaacaacccacaaaaaacgatgatctaacggaggacagcaagtccgcaacaaccttttaacagcaggctttgcggccaggagagaggaggagaggcaaagaaaaccaagcatcctccttctcccatctataaattcctccccccttttcccctctctatataggaggcatccaagccaagaagagggagagcaccaaggacacgcgactagcagaagccgagcgaccgccttctcgatccatatcttccggtcgagttcttggtcgatctcttccctcctccacctcctcctcacagggtatgtgcctcccttcggttgttcttggatttattgttctaggttgtgtagtacgggcgttgatgttaggaaaggggatctgtatctgtgatgattcctgttcttggatttgggatagaggggttcttgatgttgcatgttatcggttcggtttgattagtagtatggttttcaatcgtctggagagctctatggaaatgaaatggtttagggatcggaatcttgcgattttgtgagtaccttttgtttgaggtaaaatcagagcaccggtgattttgcttggtgtaataaagtacggttgtttggtcctcgattctggtagtgatgcttctcgatttgacgaagctatcctttgtttattccctattgaacaaaaataatccaactttgaagacggtcccgttgatgagattgaatgattgattcttaagcctgtccaaaatttcgcagctggcttgtttagatacagtagtccccatcacgaaattcatggaaacagttataatcctcaggaacaggggattccctgttcttccgatttgctttagtcccagaattttttttcccaaatatcttaaaaagtcactttctggttcagttcaatgaattgattgctacaaataatgcttttatagcgttatcctagctgtagttcagttaataggtaatacccctatagtttagtcaggagaagaacttatccgatttctgatctccatttttaattatatgaaatgaactgtagcataagcagtattcatttggattattttttttattagctctcaccccttcattattctgagctgaaagtctggcatgaactgtcctcaattttgttttcaaattcacatcgattatctatgcattatcctcttgtatctacctgtagaagtttctttttggttattccttgactgcttgattacagaaagaaatttatgaagctgtaatcgggatagttatactgcttgttcttatgattcatttcctttgtgcagttcttggtgtagcttgccactttcaccagcaaagttc

SEQ ID NO: 40 - DNA, 1244 bp, Oryza sativa
aaaaccaccgagggacctgatctgcaccggttttgatagttgagggacccgttgtgtctggttttccgatcgagggacgaaaatcggattcggtgtaaagttaagggacctcagatgaacttattccggagcatgattgggaagggaggacataaggcccatgtcgcatgtgtttggacggtccagatctccagatcactcagcaggatcggccgcgttcgcgtagcacccgcggtttgattcggcttcccgcaaggcggcggccggtggccgtgccgccgtagcttccgccggaagcgagcacgccgccgccgccgacccggctctgcgtttgcaccgccttgcacgcgatacatcgggatagatagctactactctctccgtttcacaatgtaaatcattctactattttccacattcatattgatgttaatgaatatagacatatatatctatttagattcattaacatcaatatgaatgtaggaaatgctagaatgacttacattgtgaattgtgaaatggacgaagtacctacgatggatggatgcaggatcatgaaagaattaatgcaagatcgtatctgccgcatgcaaaatcttactaattgcgctgcatatatgcatgacagcctgcatgcgggcgtgtaagcgtgttcatccattaggaagtaaccttgtcattacttataccagtactacatactatatagtattgatttcatgagcaaatctacaaaactggaaagcaataagaaatacgggactggaaaagactcaacattaatcaccaaatatttcgccttctccagcagaatatatatctctccatcttgatcactgtacacactgacagtgtacgcataaacgcagcagccagcttaactgtcgtctcaccgtcgcacactggccttccatctcaggctagctttctcagccacccatcgtacatgtcaactcggcgcgcgcacaggcacaaattacgtacaaaacgcatgaccaaatcaaaaccaccggagaagaatcgctcccgcgcgcggcggcgacgcgcacgtacgaacgcacgcacgcacgcccaaccccacgacacgatcgcgcgcgacgccggcgacaccggccgtccacccgcgccctcacctcgccgactataaatacgtaggcatctgcttgatcttgtcatccatctcaccaccaaaaaaaaaaggaaaaaaaaacaaaacacaccaagccaaataaaagcgacaa

SEQ ID NO: 41 - DNA, 654 bp, Oryza sativa
cttctacatcggcttaggtgtagcaacacgactttattattattattattattattattattattttacaaaaatataaaatagatcagtccctcaccacaagtagagcaagttggtgagttattgtaaagttctacaaagctaatttaaaagttattgcattaacttatttcatattacaaacaagagtgtcaatggaacaatgaaaaccatatgacatactataattttgtttttattattgaaattatataattcaaagagaataaatccacatagccgtaaagttctacatgtggtgcattaccaaaatatatatagcttacaaaacatgacaagcttagtttgaaaaattgcaatccttatcacattgacacataaagtgagtgatgagtcataatattatttttcttgctacccatcatgtatatatgatagccacaaagttactttgatgatgatatcaaagaacatttttaggtgcacctaacagaatatccaaataatatgactcacttagatcataatagagcatcaagtaaaactaacactctaaagcaaccgatgggaaagcatctataaatagacaagcacaatgaaaatcctcatcatccttcaccacaattcaaatattatagttgaagcatagtagta

SEQ ID NO: 42 - DNA, 50 bp, Artificial sequence
  primer prm5233
ggggacaagtttgtacaaaaaagcaggcttaaacaatgaagcggaagcgg